(19) United States
(12) Patent Application Publication
(10) Pub. No.: US 2014/0011992 A1
(43) Pub. Date: Jan. 9, 2014

(54) SYNTHESIS OF ABIRATERONE AND RELATED COMPOUNDS

(71) Applicant: Crystal Pharma, S.A.U., Boecillo-Valladolid (ES)

(72) Inventors: Alfonso Perez Encabo, Valladolid (ES); Jose Angel Turiel Hernandez, Valladolid (ES); Francisco Javier Gallo Nieto, Boecillo-Valladolid (ES); Antonio Lorente Bonde-Larsen, Boecillo-Valladolid (ES); Celso Miguel Sandoval Rodriguez, Boecillo-Valladolid (ES)

(73) Assignee: CRYSTAL PHARMA, S.A.U., Boecillo-Valladolid (ES)

(21) Appl. No.: 13/721,204

(22) Filed: Dec. 20, 2012

(51) Int. Cl. C07J 43/00; C07J 1/00; C07J 41/00

(52) U.S. Cl. CPC C07J 43/003; C07J 41/0005; C07J 1/0014
USPC 540/95; 552/518; 552/502; 552/505

(57) ABSTRACT

The present invention relates to processes for obtaining abiraterone and derivatives thereof, such as abiraterone acetate, by means of a Suzuki coupling through a steroid borate of general formula (IV) or a C—C coupling through a steroid hydrazone of general formula (II), as well as to intermediates useful in said processes.

### CROSS-REFERENCE TO RELATED APPLICATIONS

The benefit of priority is hereby claimed under 35 USC 119 of the following applications: (i) European Patent Application 11382399.1 filed Dec. 23, 2011, (ii) U.S. Provisional Patent Application 61/579,997 filed Dec. 23, 2011, and (iii) U.S. Provisional Patent Application 61/602,964 filed Feb. 24, 2012. The disclosures of such European Patent Application 11382399.1, U.S. Provisional Patent Application 61/579,997, and U.S. Provisional Patent Application 61/602,964 are hereby incorporated herein by reference, in their respective entireties, for all purposes.

### FIELD OF THE INVENTION

The present invention relates to a processes for obtaining abiraterone and derivatives thereof, such as abiraterone acetate, as well as to intermediates useful in said processes.

### BACKGROUND

Abiraterone acetate [17-(3-pyridyl)-5,16-androstadien-3β-acetate] is a steroid compound which inhibits selectively and efficiently the enzyme 17-α-hydroxylase-C17-20-lyase, which catalyzes the conversion of dehydroepiandrosterone and androstenedione to testosterone. The inhibition of said enzyme causes a strong decrease of testosterone levels in the patient and therefore this drug is used in the treatment of certain hormone-dependent tumors resistant to chemotherapy such as prostate cancer. This compound has the following chemical formula:

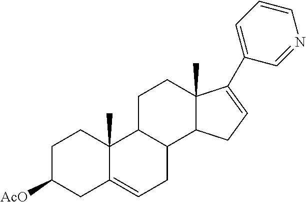 [SMILES: CC(=O)O[C@H]1CC[C@@]2(C)C(=CCC3C2CC[C@]2(C)C(c4cccnc4)=CCC32)C1]

This product was disclosed for the first time in WO 93/20097, which also provides a synthetic process for its preparation including as last step the reaction of an enol triflate with a pyridine borate by Suzuki coupling (see scheme below). However, this process is not viable in practice, mainly because of the difficulty in preparing the enol trifluorosulfonate at the 17-position 2: this step, apart from proceeding with a poor conversion and low yield, gives place to the impurity tri-unsaturated 3 in a 10% yield, which only may be removed by column chromatography. Further, the product obtained after the subsequent Suzuki coupling must be also purified by column chromatography according to the examples provided therein.

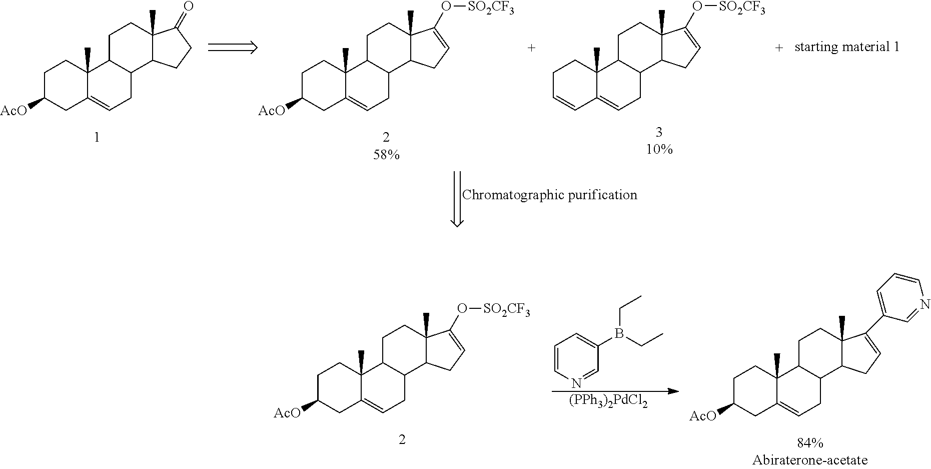

The above-mentioned impurity was prevented in later processes (EP 1 781 683 y EP 1 789 432) thanks to the use of alternative bases to that previously employed (i.e. 2,5-ditert-butyl-4-methylpyridine) such as DABCO, DBU or tryethylamine. However, in the sole example described in said documents, whilst the final product is achieved without using any column chromatography, it is obtained in a global yield of scarcely 21% and shows a purity of only 96.4%.

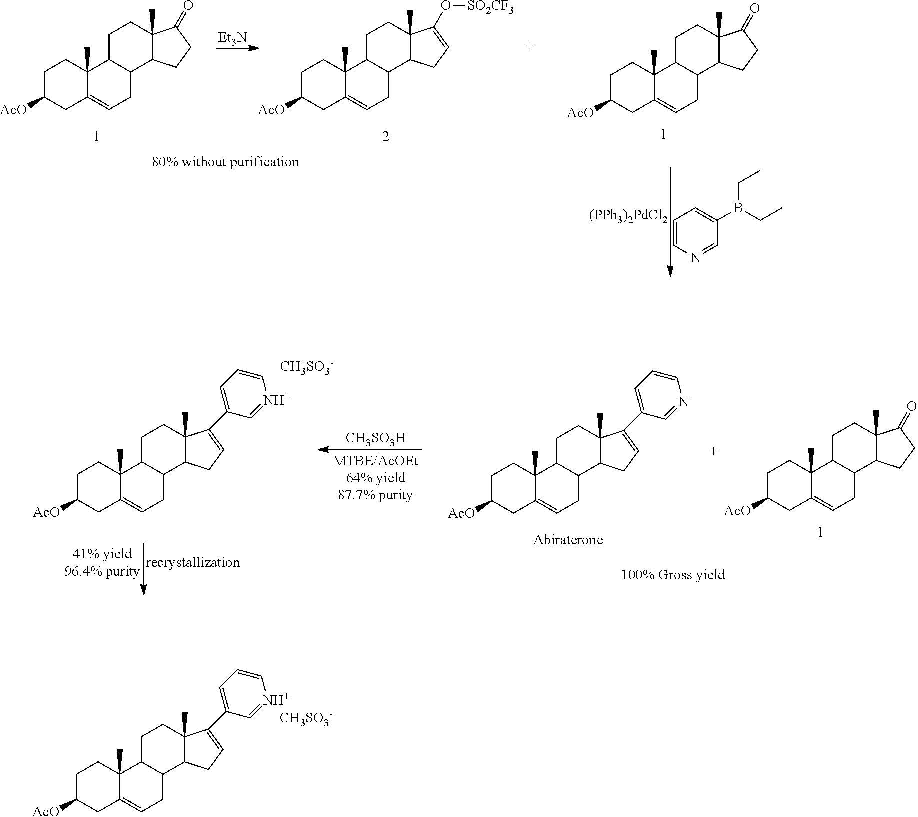

EP 0 721 461 proposes the use of a vinyl iodide or bromide intermediate instead of the enol triflate, as depicted in the following scheme:

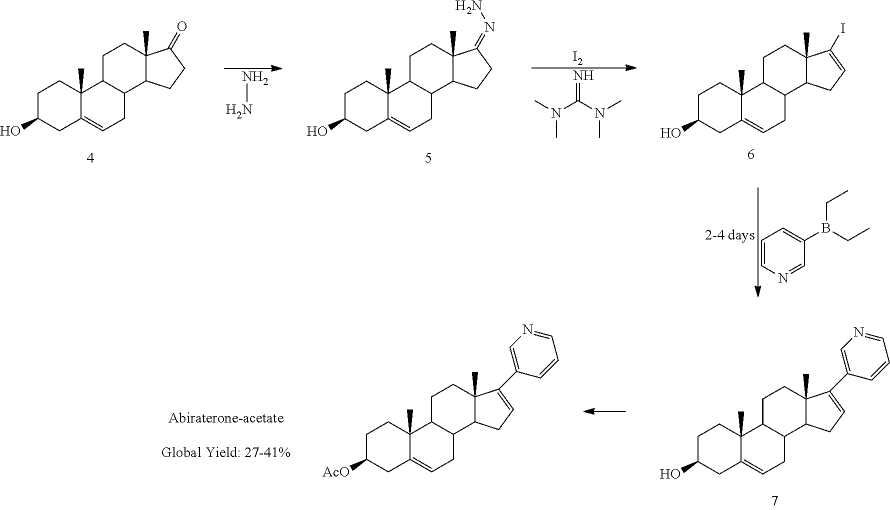

However, the iodo-enol is much less reactive than the triflate in the coupling with the pyridine borane, resulting in long reaction times (48 hours—4 days) with a part of the starting material unreacted and wherein until a 5% of a dimeric impurity is obtained, which can only be removed by purification by means of reverse phase column chromatography:

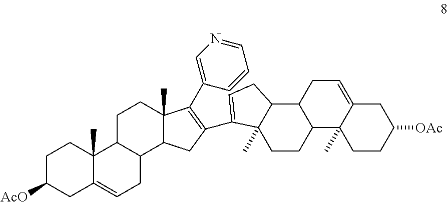 [SMILES: CC(=O)O[C@@H]1CC[C@]2(C)C(=CCC3C4CC=C(C5=C(c6cccnc6)[C@@]6(C)CCC7C(CC=C8C[C@@H](OC(C)=O)CC[C@@]87C)C6C5)[C@]4(C)CCC32)C1]

Therefore, there is still a need of developing new processes for obtaining 17-(3-pyridyl)-5,16-androstadien-3β-ol and related compounds, some of which are of therapeutic interest (e.g. abiraterone acetate) which overcome all or part of the drawbacks associated to the known processes belonging to the state of the art.

### BRIEF DESCRIPTION OF THE INVENTION

The present invention is faced with the problem of providing a process for obtaining 17-(3-pyridyl)-5,16-androstadien-3β-ol and derivatives thereof, particularly abiraterone acetate, which solves the aforementioned drawbacks existing in the different synthesis of the state of the art.

The present invention provides novel synthetic processes for obtaining abiraterone and derivatives thereof [encompassed under formula (I)], enabling its preparation at an industrial scale in an advantageous way with respect to the processes disclosed so far.

In one aspect, the present invention refers to a process for the preparation of a compound of formula (I) or a salt or solvate thereof, which comprises reacting a compound of formula (IV) with a compound of formula (III) in the presence of a palladium catalyst and a base.

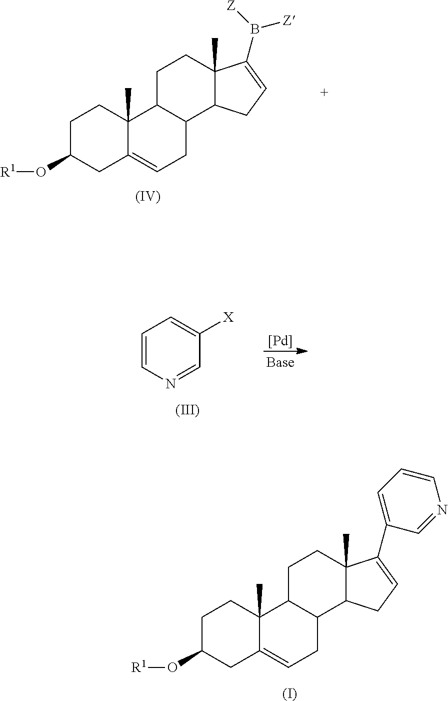

wherein X, R¹, Z and Z′ take the meaning indicated hereinafter.

In another aspect, the present invention refers to a process for the preparation of a compound of general formula (I) or a salt or solvate thereof, which comprises reacting a compound of general formula (II) with a compound of general formula (III) in presence of a palladium catalyst and a base.

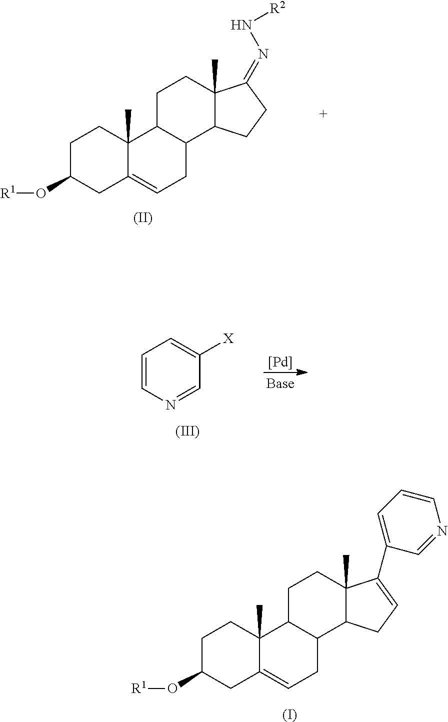

wherein X, R¹ and R² take the meaning indicated hereinafter.

The present invention also refers to a process which further comprises transformation and/or purification of a compound of formula (I) obtained by the above process into another compound of formula (I) (especially abiraterone acetate) by any known process in the state of the art, preferably, through all or some of the following steps:
i) purification of a compound of formula (I) by means of crystallization and/or salt formation;
ii) transformation of a compound of formula (I) wherein R¹ is a hydroxyl protecting group into abiraterone (R¹═H), by means of a deprotection reaction which, depending on the nature of group R¹, can comprise:
   a) hydrolysis in acid or basic media,
   b) use of fluoride reagents, or
   c) oxidation or reduction;
iii) esterification of abiraterone (R¹═H) to afford abiraterone acetate (R¹═Ac).

In a further aspect, the present invention refers to compounds of formula (II), (IV), (V), (IX), salts or solvates thereof. In a further aspect, the present invention refers to compounds of formula (I), wherein R¹ is SiR³R⁴R⁵, or a salt or solvate thereof.

In a further aspect, the present invention refers to a process for the preparation of a salt of a compound of formula (I), wherein R¹ is SiR³R⁴R⁵, by recovering the salt from a solution of the free base in any suitable solvent by treating the solution with an appropriate acid. Preferably the acid is hydrochloric acid.

These aspects and preferred embodiments thereof are additionally also defined hereinafter in the detailed description, as well as in the claims.

### DETAILED DESCRIPTION OF THE INVENTION

### Definitions

In the context of the present invention, the following terms have the meaning detailed below.

As used herein, the term “C₁-C₈ alkyl” relates to a radical derived from a linear or branched alkane, with 1 to 8 carbon atoms, for example, methyl, ethyl, propyl, butyl, etc., optionally substituted with one or more substituents independently selected from halogen, C₆-C₁₄ aryl and C₁-C₈ alkyl. An example of substituted alkyl is benzyl, which may be, in turn, substituted with methoxy, nitro, cyano, halo, phenyl, etc.

As used herein, the term “C₃-C₆ cycloalkyl” relates to a radical derived from a cycloalkane, with 3 to 6 carbon atoms, for example, cyclopropyl, cyclobutyl, etc., optionally substituted with one or more substituents independently selected from halogen, C₆-C₁₄aryl and C₁-C₈ alkyl.

As used herein, the term “C₆-C₁₄ aryl” relates to a radical derived from an aromatic hydrocarbon, with 6 to 14 carbon atoms, for example, phenyl, tolyl, xylyl, naphthyl, etc., optionally substituted with one or more substituents independently selected from halogen and C₁-C₈ alkyl.

As used herein, the term “C₁-C₈ alkoxy” relates to an O-alkyl radical, with 1 to 8 carbon atoms, for example, methoxy, ethoxy, propoxy, butoxy, etc., optionally substituted with one or more substituents independently selected from halogen and C₁-C₈ alkyl.

As used herein, the term “C₂-C₃ alkylenedioxy” is a divalent group represented by —O—R—O—, where R is an alkylene group of two or three carbon atoms optionally substituted with one or more substituents independently selected from C₆-C₁₄ aryl and C₁-C₈ alkyl. Examples of alkylenedioxy groups include —O—CH₂—CH₂—O—, —O—C(CH₃)₂—C(CH₃)₂—O—, —O—C(CH₃)₂—CH(CH₃)—O—, —O—CH₂—CH₂—CH₂—O—, —O—CH₂—C(CH₃)₂—CH₂—O— and —O—C(CH₃)₂—CH₂—CH(CH₃)—O—.

As used herein, the term “C₆ aryldioxy” is a divalent group represented by —O—R—O—, where R is an aryl group of six carbon atoms optionally substituted with one or more substituents independently selected from C₁-C₈ alkyl. Preferably, it is benzene-1,2-dioxy.

As used herein, the term “halogen” or “halo” relates to fluorine, chlorine, bromine or iodine.

As used herein, the term “hydroxyl protecting group” (HPG) includes any group capable of protecting a hydroxyl group. Illustrative examples of hydroxyl protecting groups have been described by Green T W et al. in “Protective Groups in Organic Synthesis”, 3rd Edition (1999), Ed. John Wiley & Sons (ISBN 0-471-16019-9). Virtually any hydroxyl protecting group can be used to put the invention into practice. Nevertheless, in a particular embodiment, the hydroxyl protecting group is an ester group or an ether group, which can be converted into a hydroxyl group under mild conditions. Illustrative, non-limiting examples of HPGs include esters (COR), carbonates (COOR), amides (CONRR′), silyl radicals [Si(R³)(R⁴)(R⁵)], and ethers (R⁶), wherein:
R and R′ are independently selected from the group consisting of optionally substituted C₁-C₈ alkyl, optionally substituted C₃-C₆ cycloalkyl and optionally substituted C₆-C₁₄ aryl;
R³, R⁴ and R⁵ are independently selected from the group consisting of optionally substituted C₁-C₈ alkyl, optionally substituted C₃-C₆ cycloalkyl, optionally substituted C₆-C₁₄ aryl, optionally substituted C₁-C₈ alkoxy, and halogen; and
R⁶ is selected from the group consisting of optionally substituted C₁-C₈ alkyl, optionally substituted C₃-C₆ cycloalkyl and optionally substituted C₆-C₁₄ aryl.

Representative examples of esters and carbonates as HPGs are those wherein R is methyl or benzyl.

Representative examples of amides as HPGs are those wherein R and/or R′ are independently selected from methyl and benzyl.

Representative examples of silyl groups as HPGs are those wherein R³, R⁴ and R⁵ are independently selected from C₁-C₄ alkyl and C₆-C₁₄ aryl; preferably are independently selected from methyl, i-propyl, t-butyl and phenyl. More preferably, the silyl group is trimethylsilyl (TMS), dimethylphenylsilyl (DMPS) or dimethyl-t-butylsilyl (TBDMS).

Representative examples of ethers as HPGs are methyl ethers (CH₂OR⁸), ethyl ethers (CH₂CH₂OR⁸) and benzyl ethers, wherein R⁸ is C₁-C₈ alkyl, such as methoxymethyl, 1-ethoxyethyl.

The compounds used in the process described by the present invention can be obtained in free form or in solvate form. In both cases, they are preferably obtained in crystalline form, both as free compounds or as solvates (for example, hydrates, alcoholates, etc.), both forms being included within the scope of the present invention. Solvation methods are generally well known in the state of the art.

The invention also provides “salts” of the compounds described in the present description. By way of illustration, said salts can be acid addition salts, base addition salts or metal salts, and can be synthesized from the parent compounds containing a basic or acid moiety by means of conventional chemical processes known by the persons skilled in the art. Such salts are generally prepared, for example, by reacting the free acid or base forms of said compounds with a stoichiometric amount of the suitable base or acid in water or in an organic solvent or in a mixture of the two. Non-aqueous media such as ether, ethyl acetate, ethanol, acetone, isopropanol or acetonitrile are generally preferred. Illustrative examples of said acid addition salts include inorganic acid addition salts such as, for example, hydrochloride, hydrobromide, hydroiodide, sulfate, nitrate, phosphate, etc., organic acid addition salts such as, for example, acetate, maleate, fumarate, citrate, oxalate, succinate, tartrate, malate, mandelate, methanesulfonate, p-toluenesulfonate, camphorsulfonate, etc. Illustrative examples of base addition salts include inorganic base salts such as, for example, ammonium salts and organic base salts such as, for example, ethylenediamine, ethanolamine, N,N-dialkylenethanolamine, triethanolamine, glutamine, amino acid basic salts, etc. Illustrative examples of metal salts include, for example, sodium, potassium, calcium, magnesium, aluminum and lithium salts.

The term “pharmaceutically acceptable” relates to molecular entities and compositions being physiologically tolerable and normally not causing an allergic reaction or similar adverse reaction, such as gastric discomfort, dizziness and the like, when they are administered to a human being. Preferably, as used in this description, the term “pharmaceutically acceptable” means approved by a governmental regulatory agency or listed in the US pharmacopoeia or another generally recognized pharmacopoeia for use in animals, and more particularly in humans.

For those persons skilled in the art, it will be evident that the scope of the present invention also includes salts which are not pharmaceutically acceptable as possible means for obtaining pharmaceutically acceptable salts.

Unless otherwise indicated, the compounds of the invention also include compounds which differ in the presence of one or more isotopically enriched atoms. By way of illustration, compounds having the structures defined herein, with the exception of the substitution of at least one hydrogen by a deuterium or tritium, or the substitution of at least one carbon by a carbon enriched in ¹³C or ¹⁴C, or at least one nitrogen by a nitrogen enriched in ¹⁵N, are within the scope of this invention.

The term “complex” means a molecular structure in which neutral molecules or anions (called ligands) bond to a central metal atom (or ion) by coordinate covalent bonds. Extensive descriptions of terms related to coordination chemistry in reference books such as Robert H. Crabtree “The Organometallic Chemistry of the Transition Metals”, Wiley-Interscience; 4 ed., 2005.

The term “catalyst” is recognized in the art and means a substance that increases the rate of a reaction without modifying the overall standard Gibbs energy change in the reaction and without itself being consumed in the reaction. The changing of the reaction rate by use of a catalyst is called catalysis. As used herein, the catalyst is used in a substoichiometric amount relative to a reactant, i.e. a catalytic amount. A preferred catalytic amount is considered herein from 0.001 to 20 mol % of catalyst relative to the steroid compound to undergo coupling.

The term “ligand” refers to a molecule or ion that is bonded directly (i.e. covalently) to a metal center.

As used herein, the term “about” means a slight variation of the value specified, preferably within 10 percent of the value specified. Nevertheless, the term “about” can mean a higher tolerance of variation depending on for instance the experimental technique used. Said variations of a specified value are understood by the skilled person and are within the context of the present invention. Further, to provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

### Suzuki Coupling Reaction Through a Organoboron Steroid Compound (IV)

After an extensive research on processes for the preparation of abiraterone and related compounds, the inventors have surprisingly found that it is easier and more convenient to carry out a Suzuki type coupling, in which the organoboron compound is situated on the steroid and the electrophilic compound is the 3-substituted pyridine, conversely to the processes disclosed previously in the state of the art.

Therefore, in one aspect, the present invention is directed to the preparation of a compound of formula (I) or a salt or solvate thereof, by reacting a compound of formula (IV) with a 3-substituted pyridine of formula (III) in presence of a Pd(0) or Pd(II) catalyst and a base

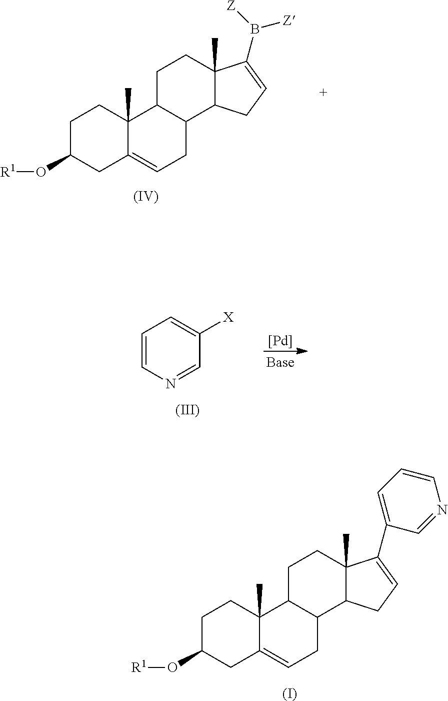

wherein
R¹ is selected from the group consisting of H and a hydroxyl protecting group (HPG);
X is halogen or OSO₂CF₃; and
Z and Z′ are independently selected from the group consisting of hydroxyl, optionally substituted C₁-C₈ alkoxy and optionally substituted C₁-C₈ alkyl, or Z and Z′ together form an optionally substituted C₂-C₃ alkylenedioxy group or an optionally substituted C₆ aryldioxy group.

The reaction of the compounds of formula (IV) with the compounds of formula (III) is carried out under Pd catalysis, for example in presence of a Pd(0) catalyst such as Pd(PPh₃)₄ or Pd₂(dba)₃ or a Pd(II) catalyst, which is reduced in situ to Pd(0) such as Pd(OAc)₂, Pd(PPh₃)₂Cl₂, Pd(dppe)₂Cl₂ (dppe=(1,2-bis(diphenylphosphino)ethane), Pd(dppf)Cl₂ (dppf=1,1′-bis(diphenylphosphino)ferrocene), Pd(dppf)Cl₂.CH₂Cl₂, Pd(dcypp)Cl₂ (dcypp=bis(dicyclohexylphosphino)propane), Pd(PhCN)₂Cl₂ or Pd(CH₃CN)₂Cl₂. Preferably, the Pd catalyst is a Pd(0) or a Pd(II) catalyst having phosphine ligands such as Pd(PPh₃)₄, Pd(PPh₃)₂Cl₂, Pd(dppe)₂Cl₂, Pd(dppf)Cl₂ or Pd(dppf)Cl₂.CH₂Cl₂. More preferably, it is Pd(PPh₃)₄ or Pd(dppf)Cl₂.CH₂Cl₂. Typically, the amount of the Pd catalyst is from about 0.001% mol to about 6% mol, such as from about 0.01% mol to about 6% mol. In some embodiments, the mount of the Pd catalyst is from about 0.2% mol to about 6% mol, preferably about 0.5-2% mol.

The process requires the presence of a base. Suitable bases include alkaline and alkaline earth metal carbonates, bicarbonates, phosphates, acetates, alkoxides, hydroxides and halides. Preferably, the base is an alkaline metal carbonate or an alkaline earth metal carbonate. More preferably, the base is selected from sodium, cesium, potassium and calcium carbonate. Even more preferably, it is sodium carbonate, potassium carbonate or calcium carbonate.

Further, in a particular embodiment the reaction proceeds in the presence of water either in a homogenous system or a biphasic system.

According to a particular embodiment, this coupling reaction is carried out in the presence of an organic solvent or mixture of solvents, for example, an ether (e.g., tetrahydrofuran (THF), 2-methyltetrahydrofuran, 1,2-dimethoxyethane (DME), dioxane, 1,3-dioxolane, etc.) or an aromatic solvent (e.g., toluene, xylene, etc.) or mixtures thereof. In a particular embodiment, the reaction of the compounds of formula (IV) and (III) is carried out in a mixture of organic solvents, preferably THF and toluene, in the presence of variable amounts of water; the amount of water typically ranges from about 10% to about 100% with respect to the amount of the organic solvent/s employed. In a particular embodiment, the amount of water ranges from about 2% to about 50%, preferably from about 10% to about 30%, with respect to the amount of the organic solvent/s employed. In another particular embodiment, the reaction of the compounds of formula (IV) and (III) is carried out in THF in the presence of water.

Likewise, the coupling reaction is suitably carried out under heating, for example at temperatures comprised between about 40° C. and about 110° C., preferably between about 60° C. and about 90° C. or at the boiling point temperature.

The compound of formula (III) is typically used in an amount ranging from about 1.0 and about 3.0 equivalents for each equivalent of compound of formula (IV), preferably from about 1.2 to about 1.6 equivalents.

In a particular embodiment, X is selected from bromo, iodo and OSO₂CF₃. A particular and preferred example of compound of formula (III) is 3-bromopyridine.

In a particular embodiment, R¹ is selected from H and a silyl protecting group of formula Si(R³)(R⁴)(R⁵). Preferably, R³, R⁴ and R⁵ are independently selected from C₁-C₄ alkyl and C₆-C₁₄ aryl; more preferably are independently selected from methyl, i-propyl, t-butyl and phenyl. In a further embodiment, R¹ is trimethylsilyl (TMS), dimethylphenylsilyl (DMPS) or dimethyl-t-butylsilyl (TBDMS). Preferably, R¹ is TBDMS.

According to a particular embodiment, in the compound of formula (IV) R¹ is selected from the group consisting of H, COMe, SitBuMe₂ (TBDMS) and SiPhMe₂ (DMPS). Preferably, R¹ is TBDMS.

According to another particular embodiment, in the compound of formula (IV) Z and Z′ are OH, methoxy, ethoxy, i-propoxy or, together, form an ethylendioxy, tetramethylethylenedioxy, propylendioxy, dimethylpropylendioxy, trimethylpropylendioxy, tetramethylpropylendioxy or benzene-1,2-dioxy group. Preferably, Z and Z′ are OH or ethoxy. More preferably, Z and Z′ are OH.

Depending on the R¹ group and the reaction conditions, radical R¹ in the compound of formula (I) can be the same or different from radical R¹ in the starting compound of formula (IV). In a particular embodiment, R¹ in the compound of formula (IV) is a HPG which is cleaved under coupling reaction conditions giving rise to a compound of formula (I) wherein R¹ is OH. In another embodiment, the HPG is not cleaved under coupling reaction conditions and, therefore, R¹ is a HPG in the compound of formula (IV) and in the resulting compound of formula (I).

According to a particular embodiment, R¹ is the same in the compound of formula (IV) and in the compound of formula (I) and is selected from the group consisting of H, COMe, SitBuMe₂ (TBDMS) and SiPhMe₂ (DMPS). More preferably, R¹ is TBDMS.

In a particular embodiment, a compound of formula (I) wherein R¹ is TBDMS is obtained by reacting a compound of formula (IV) wherein R¹ is TBDMS and Z and Z′ are OH with 3-bromopyridine. Preferably, this reaction is performed in the presence of Pd(PPh₃)₄ or Pd(dppf)Cl₂.CH₂Cl₂ as catalyst and Na₂CO₃, K₂CO₃ or CaCO₃ as base. Also preferably, this reaction is performed in the presence of a mixture of THF, toluene and water, or in the presence of a mixture of THF and water or in the presence of water.

In a preferred embodiment, a compound of formula (I) wherein R¹ is TBDMS is obtained by reacting a compound of formula (IV) wherein R¹ is TBDMS and Z and Z′ are OH with 3-bromopyridine, in the presence of 6% mol Pd(PPh₃)₄ and 1.5 equivalents of Na₂CO₃ and a mixture of THF, toluene and water.

In another preferred embodiment, a compound of formula (I) wherein R¹ is TBDMS is obtained by reacting a compound of formula (IV) wherein R¹ is TBDMS and Z and Z′ are OH with 3-bromopyridine, in the presence of Pd(dppf)Cl₂.CH₂Cl₂ and carbonate as a base and water.

In another preferred embodiment, a compound of formula (I) wherein R¹ is TBDMS is obtained by reacting a compound of formula (IV) wherein R¹ is TBDMS and Z and Z′ are OH with 3-bromopyridine, in the presence of Pd(dppf)Cl₂.CH₂Cl₂ and CaCO₃ and a mixture of THF and water.

Once the reaction is complete, inorganic salts may be removed by washing with water and extracting with an organic solvent such as dichloromethane, toluene, diethyl ether, cyclopentylmethyl ether, ethyl acetate or any other suitable solvent.

The product contained in the organic phase can, depending on the nature of R¹, be isolated by solvent removal and recrystallization of the residue in a suitable solvent, or alternatively, by precipitation in the form of a salt, such as chlorhydrate, bromhydrate, sulfate, methanesulfonate, malate, etc., or a combination of both methods. Dilute solutions of acid will be preferably used when R¹ is protected with certain HPGs such as silyl radicals in order to avoid the cleavage of the protecting group.

Alternatively, once the reaction is complete, the product can be isolated by solvent removal followed by addition of an HCl aqueous solution in order to precipitate the product as a hydrochloride salt and isolate it, for example, by filtration.

### C—C Coupling Through a Hydrazone of General Formula (II)

Palladium-catalyzed C—C coupling reactions usually require the presence of a component with nucleophilic character represented by an organometallic compound such as organoboron (Suzuki), organozinc (Negishi), organostannane (Stille), etc. Recently, it has been found that certain hydrazones, in presence of a base, can act as nucleophiles in the same way without requiring any organometallic specie (A. Takemiya, J. F. Hartwig, J. Am. Chem. Soc., 2006, 128, 14800).

More recently, this methodology has been applied to the preparation of di- and tri-substituted olefins from tosyl-hydrazones in presence of a base, an aryl halide and a Pd catalyst (J. Barluenga et al, Angew. Chem. Int. Ed., 2007, 46, 5587-90 and J. Barluenga et al, Chem. Eur. J. 2008, 14, 4792-5).

In this context, the inventors have surprisingly and unexpectedly found that it is possible to extend this methodology to the preparation of vinyl-aryl(heteroaryl) derivatives such as the compounds of general formula (I), among which abiraterone is encompassed, by reaction of a steroid hydrazone of general formula (II) with a 3-substituted pyridine of general formula (III) in presence of a Pd(0) or Pd(II) catalyst and a base

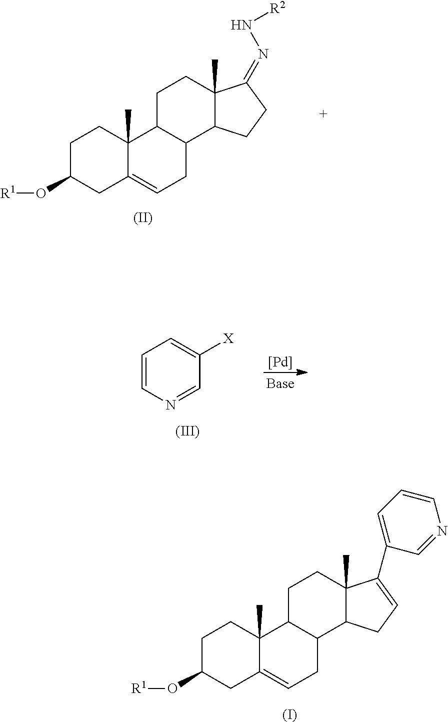

wherein
R¹ is selected from the group consisting of H and a hydroxyl protecting group (HPG);
R² is SO₂R⁷;
R⁷ is selected from the group consisting of optionally substituted C₁-C₈ alkyl and optionally substituted C₆-C₁₄ aryl; and
X is halogen or OSO₂CF₃.

As compared with the prior art approaches, neither the enolization of the ketone group at the 17-position nor the use of boron organometallic reagents are necessary in this process. Further, the impurities disclosed in the background of the invention for the prior procedures are not obtained, thus avoiding their purification. All this results in a cleaner and straighter process with better yields than those described so far for obtaining this kind of substrates. Moreover, as will be described hereinafter, it is possible to arrive at abiraterone and related compounds of general formula (I) from the starting ketone steroids without isolation of intermediate compounds, i.e., in a one-pot fashion.

The reaction of the compounds of general formula (II) with the compounds of general formula (III) is carried out under Pd catalysis. Examples of Pd catalysts that may be used include, without limitation, [Pd₂(dba)₃] (dba=trans, trans-dibenzylidene acetone), Pd(PPh₃)₄, Pd(dppf)Cl₂.CH₂Cl₂(dppf=1,1′-bis(diphenylphosphino)ferrocene), Pd(dcypp)Cl₂ (dcypp=bis(dicyclohexylphosphino)propane), PdCl₂(CNMe)₂, Pd(OH)₂ and Pd(OAc)₂ (Organic Letters 2010, 12 (18), 4042-4045). Preferably the catalyst is selected from Pd₂(dba)₃, Pd(PPh₃)₄, Pd(dppf)Cl₂.CH₂Cl₂ and PdCl₂(CNMe)₂, more preferably the Pd catalyst is Pd₂(dba)₃ or Pd(dppf)Cl₂.CH₂Cl₂. Typically, the amount of the catalyst ranges from about 0.5% mol to about 10% mol, preferably from about 1% mol to about 6% mol.

Together to the Pd catalyst, ligands able to associate with the Pd atom have been used to facilitate the reaction so that a palladium complex is formed in the reaction media. In a preferred embodiment, the ligand is a phosphine ligand. Phosphine ligands are widely known by the skilled person since they are commonly used in organic catalysis. Illustrative, non-limiting examples of suitable ligands include X-phos (2-dicyclohexylphosphino-2′,4′,6′-triisopropylbiphenyl), dppp (1,4-bis(diphenylphosphino)butane), S-phos (2-dicyclohexylphosphino-2′,6′-dimethoxybiphenyl), dppm (1,1-bis(diphenylphosphino)-methane), dippe (1,2-bis(diisopropylphosphino)ethane, dmpe (1,2-Bis(dimethylphosphino)ethane, dppe (1,2-bis(diphenylphosphino)ethane, etc. Preferably the ligand is X-phos or dppp, more preferably X-phos. The amount of ligand used depends on the amount of palladium catalyst. In general, the amount of the ligand ranges from about 1% mol to about 20% mol, preferably from about 2% mol to about 12% mol.

The coupling is performed in basic medium. Alkoxides and carbonates of alkaline and alkaline earth metals have been found particularly useful such as for example alkoxides and carbonates of Li, Na, K and Cs. Illustrative, non-limiting examples of suitable bases for the coupling step include t-BuOLi, MeOLi, MeONa and CsCO₃. In a preferred embodiment, the base is t-BuOLi. The amount of base preferably ranges from about 2 to about 20 eq for each equivalent of compound of formula (II), more preferably from about 4 to about 15 eq. In general, about 7.5 eq of base are suitable for the reaction occurs.

According to a particular embodiment, this coupling reaction is carried out in an organic solvent or mixture of solvents, for example, an ether (e.g., tetrahydrofuran (THF), 2-methyltetrahydrofuran, 1,2-dimethoxyethane (DME), dioxane, 1,3-dioxolane, etc.) or an aromatic solvent (e.g., toluene, xylene, etc.). In a more particular embodiment, the reaction of the compounds of formulas (II) and (III) is carried out in dioxane.

Likewise, the coupling reaction is suitably carried out under heating, for example at temperatures comprised between about 40° C. and about 140° C., preferably between about 65° C. and about 110° C., more preferably between about 80° C. and about 110° C.

The compound of formula (III) is typically used in an amount ranging from about 1.1 and about 3 eq for each equivalent of compound of formula (II), preferably from about 1.3 to about 1.6 eq. A particular and preferred example of compound of formula (III) is 3-bromopyridine.

In a particular embodiment, R¹ is selected from H and a silyl protecting group of formula Si(R³)(R⁴)(R⁵). Preferably, R³, R⁴ and R⁵ are independently selected from C₁-C₄ alkyl and C₆-C₁₄ aryl; more preferably are independently selected from methyl, i-propyl, t-butyl and phenyl. In a further embodiment, R¹ is trimethylsilyl (TMS), dimethylphenylsilyl (DMPS) or dimethyl-t-butylsilyl (TBDMS). Preferably, R¹ is TBDMS.

According to a particular embodiment, in the compound of formula (II) R¹ is selected from the group consisting of H, COMe, SitBuMe₂ (TBDMS) and SiMe₂Ph (DMPS). More preferably, R¹ is TBDMS.

According to another particular embodiment, in the compound of formula (II) R² is SO₂R⁷ wherein R⁷ is an optionally substituted C₆-C₁₄ aryl, such as Ph, Tol, 2,4,6-trimethylphenyl or 2,4,6-triisopropylphenyl.

The products of formula (I) obtained can be purified by column chromatography or preferably by means of industrially acceptable processes such as, for example, by means of a crystallization process, either of the product as a free base or, more preferably, through the formation of an addition salt (e.g. chlorhydrate, bromhydrate, sulfate, methanesulfonate, malate, etc). During the process of precipitation as a salt, the product is purified from all those impurities of neutral character. Illustrative, non-limiting examples of suitable solvents for said crystallization are THF, ethyl acetate and isopropyl ether. Likewise, addition salts of HCl and malic acid are preferred. Dilute solutions of acid will be preferably used when R¹ is protected with certain HPGs such as silyl radicals in order to avoid the cleavage of the protecting group.

In a especially preferred embodiment, 3-(tert-butyldimethylsiloxy)-17-p-toluenesulfonylhydrazone-androsta-5-en is subject to reaction with 6% mol Pd₂(dba)₃, 12% mol X-Phos, 7.5 eq tBuOLi and 1.5 eq 3-bromopyridine in 20-30 volume of dioxane at a temperature between 90 and 100° C. for 6-15 hours. Then, the reaction mixture is filtered and the solvent evaporated. The residue obtained is purified by re-dissolution in THF and precipitation of the product obtained as an acid addition salt of HCl (using HCl aq 1-2M) at room temperature. Thus, the salt obtained can be used directly or can be neutralized to obtain the product in neutral form. This process for obtaining chlorhydrate of 3-(tert-butyldimethylsiloxy)-17-(3-pyridyl)-androsta-5-en is depicted below:

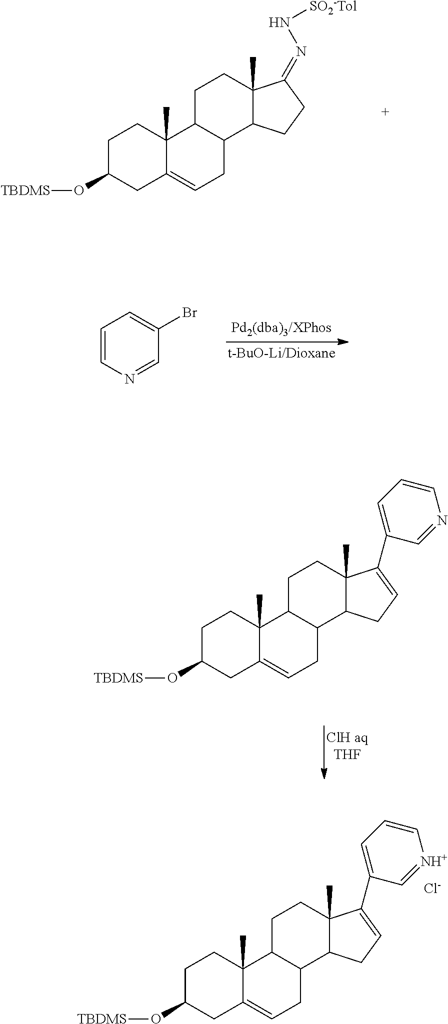

The compounds of general formula (II) can be readily prepared from a ketone of general formula (VI) and a hydrazine of general formula (VII):

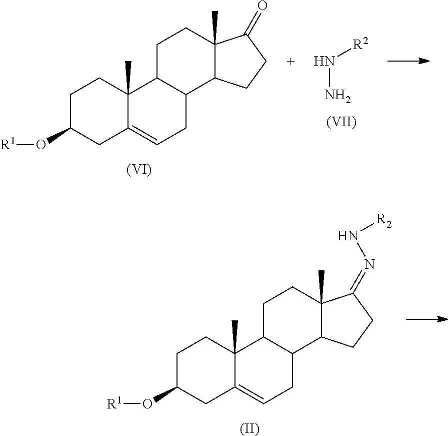

This reaction can be conveniently carried out by mixing both components in equimolecular amounts or with an excess of hydrazine (e.g. about 10%), preferably at 40° C.-110° C., in a suitable solvent. From an experimental point of view, it is preferred to use the same solvent than that employed in the subsequent step such as dioxane, THF, etc., (J. Barluenga et al, Chem. Eur. J. 2008, 14, 4792-5). Further, the reaction can be catalyzed by using about 0.05-0.1 eq TsOH or any other suitable acid which speeds the reaction. Under catalysis, the process typically undergoes in about two hours at 50-90° C.

The hydrazones thus obtained, optionally, can be isolated by precipitation by addition over water or by solvent evaporation and precipitation of the residue by addition of diethyl ether, toluene, heptane or any other suitable solvent, depending on the nature of R¹.

It has been found that the condensation of a ketone of general formula (VI) and a hydrazine of general formula (VII), depending on the nature of R¹, may proceed with the hydrolysis of the hydroxyl protecting group on position 3 to some extent. Accordingly, in some instances, said condensation is initially carried out with 5-dehydroepiandrosterone (5-DHEA) to obtain the corresponding hydrazone (II-H), which may be subsequently protected on its hydroxyl group through common processes known in the state of the art (see scheme below).

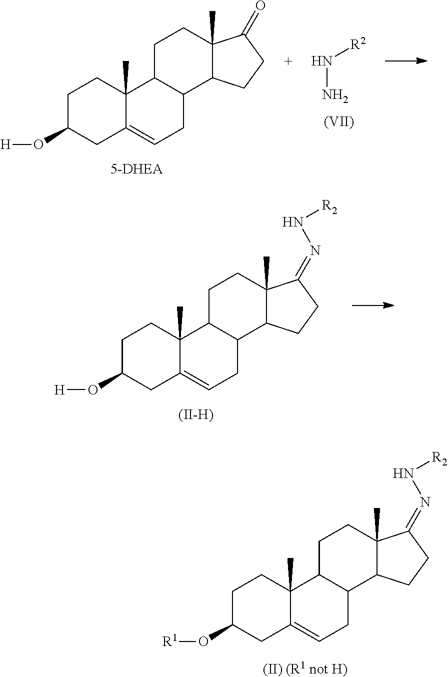

According to a preferred embodiment, the formation of the hydrazone of general formula (II) may be carried out in THF with 0.1 eq of TsOH and 1.1 eq of p-toluenesulfonyl hydrazide at 50° C.-90° C. After completion, the reaction mixture is poured into water under stirring to afford a solid, which is subsequently filtered.

The easiness of this process opens the possibility that the compounds of general formula (I) can be also obtained directly from the compounds of general formula (VI), without isolation of the intermediate compounds (II), i.e. in a one-pot fashion, as depicted below.

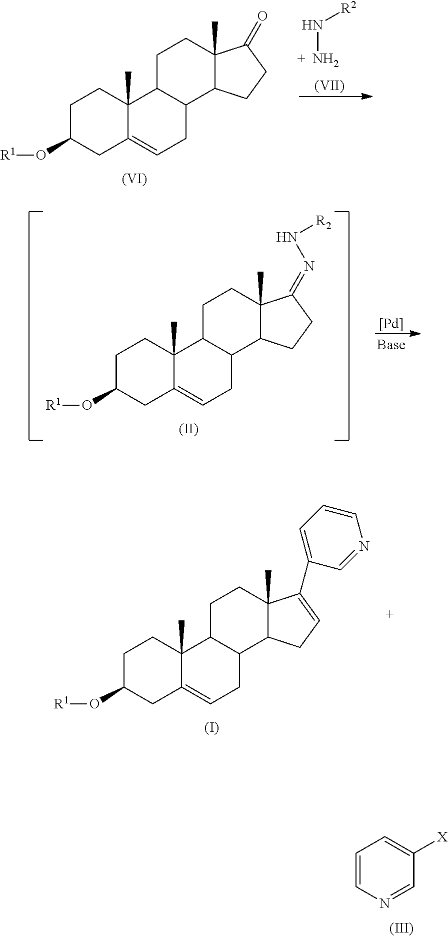

Transformation and/or Purification of Compounds of Formula (I)

The processes of the invention may further comprise the transformation and/or purification of the compounds of formula (I) by any known process in the state of the art, preferably through some or all of the following steps:
i) purification of a compound of formula (I) by means of crystallization and/or salt formation;
ii) transformation of a compound of formula (I) wherein R¹ is a hydroxyl protecting group into abiraterone (R¹═H), by means of a deprotection reaction which, depending on the nature of group R₁, can comprise:
   a) hydrolysis in acid or basic media,
   b) use of fluoride reagents, or
   c) oxidation or reduction;
iii) esterification of abiraterone (R¹═H) to afford abiraterone acetate (R¹═Ac).
   Step i) Purification of a Compound of Formula (I) by Means of Crystallization and/or Salt Formation.

The compounds of formula (I) obtained can be purified by any conventional method, such as by column chromatography or, more preferably, by means of industrially acceptable processes such as, for example, by means of a crystallization process, either of the product as a free base or, more preferably, through the formation of an addition salt (e.g. chlorhydrate, bromhydrate, sulfate, methanesulfonate, malate, etc.). During the process of precipitation as a salt, the product can be purified from impurities of neutral character. Illustrative non-limiting examples of suitable solvents for said crystallization are THF, ethyl acetate and isopropyl ether. Likewise, addition salts of HCl and malic acid are preferred. The salt obtained in this way may be used directly or may be neutralized.

Particular compounds of formula (I) which may be isolated as a chlorhydrate salt by precipitation after addition of an aqueous solution of chloride acid are those wherein R¹ is selected from H, COMe, TMS, DMPS and TBDMS.

Step ii) Transformation of a Compound of Formula (I) Wherein R¹ is a Hydroxyl Protecting Group into Abiraterone (R¹═H).

Abiraterone can be prepared from a compound of formula (I) wherein R¹ is a hydroxyl protecting group by conventional methods of deprotection known by persons skilled in the art (Green T W et al. in “Protective Groups in Organic Synthesis”, 3rd Edition (1999), Ed. John Wiley & Sons (ISBN 0-471-16019-9).

For example, compounds of formula (I) wherein R¹ represents an ester (COR), a carbonate (COOR) or an amide (CONRR′) can be easily converted into abiraterone by hydrolysis in basic or acid media according to well-established procedures of the state of the art.

Compounds of formula (I) wherein R¹ represents a silyl radical (SiR³R⁴R⁵) can be easily converted into abiraterone by the use of fluoride reagents such as fluoride salts or HF, acid media, oxidizing media, etc.

According to a particular embodiment, a compound of formula (I) wherein R¹ is a silyl group, preferably TBDMS, is transformed into abiraterone by treatment with tetrabutylammonium fluoride in the presence of an organic solvent. According to a more particular embodiment, when R¹=TBDMS, the deprotection conditions are: the silyl derivative is solved in THF and tetrabutylammonium fluoride in THF (1 M) is added at room temperature. The progress of this reaction can be easily monitored by TLC. The alcohol is isolated.

Compounds of formula (I) wherein R¹ represents an ether (R⁶) can be easily converted into abiraterone through hydrolysis in acid media (for example, for methyl ethers (CH₂OR⁸)), hydrogenation (for example, for benzyl ethers), oxidation (for example, for aryl ethers), etc.

Step iii) Esterification of Abiraterone (R¹═H) to Afford Abiraterone Acetate (R¹═Ac).

The esterification of abiraterone into its acetate may be performed according to conventional chemical processes known by those skilled in the art. According to a particular embodiment, this esterification is carried out by using acetyl chloride as acylating agent and pyridine as solvent, or acetyl chloride as acylating agent and ethyl ether as solvent in the presence of DMAP as catalyst (EP 0721461 B, U.S. Pat. No. 5,618,807 A).

In a particular embodiment, a compound of formula (I) wherein R¹ is TBDMS is obtained by reacting a compound of formula (IV) wherein R¹ is TBDMS and Z and Z′ are OH with 3-bromopyridine, which is further transformed into a compound of formula (I) wherein R¹ is H by deprotection of the hydroxyl group, preferably in the presence of a fluoride reagent such as tetrabutylammonium fluoride. In a particular embodiment, this process further comprises esterification of abiraterone to abiraterone acetate, preferably in the presence of acetyl chloride.

Purification of compounds of formula (I) can be performed at any stage of the synthesis, i.e. before and/or after transformation into abiraterone and/or before and/or after transformation into abiraterone acetate.

### Synthesis of Intermediate Compounds of Formula (IV)

Boron derivatives of formula (IV) may be obtained from hydrazones of formula (IIa) through the following sequence (J. Am. Chem. Soc., 2008, 130, 8481-8490):

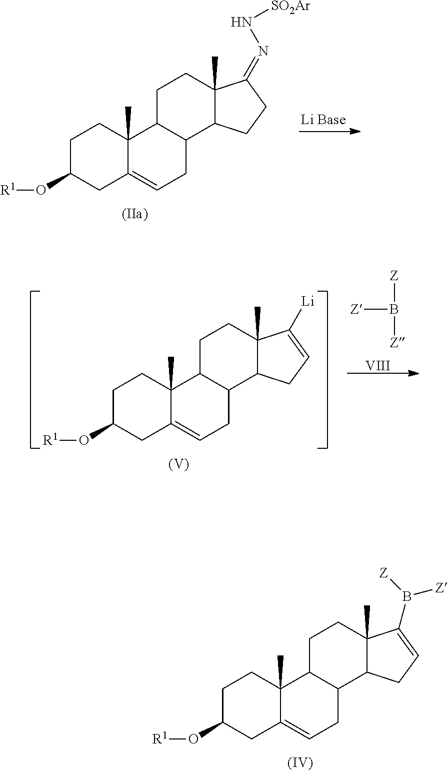

wherein:
R¹ is as previously defined,
Ar is an optionally substituted C₆-C₁₄ aryl. Preferably Ar is phenyl, tolyl, 2,4,6-trimethylphenyl or 2,4,6-triisopropylphenyl,
Z and Z′ are as previously defined, and
Z″ is selected from the group consisting of hydroxyl, optionally substituted C₁-C₈ alkoxy and optionally substituted C₁-C₈ alkyl.

Compounds of formula (IIa) may be prepared in turn from ketones of formula (VI), similarly to the compounds of formula (II) as defined above and compound of formula (IIb) as defined hereinafter.

Enol lithium compounds of formula (V) may be prepared by Shapiro reaction, from hydrazones of formula (IIa) and a lithium base, including common alkyl lithium bases such as n-BuLi, Hexyl-Li, tert-BuLi, etc. Then, the enol lithium compound is reacted with a boronic acid or boronic ester of formula (VIII) to afford a vinyl-borate of formula (IV).

Alternatively, the compound of formula (IV) may be obtained from a compound of formula (IX) by treatment with a lithium base and a compound of formula (VIII) according to the following scheme (J. Org. Chem., 1985, 50, 2438-43):

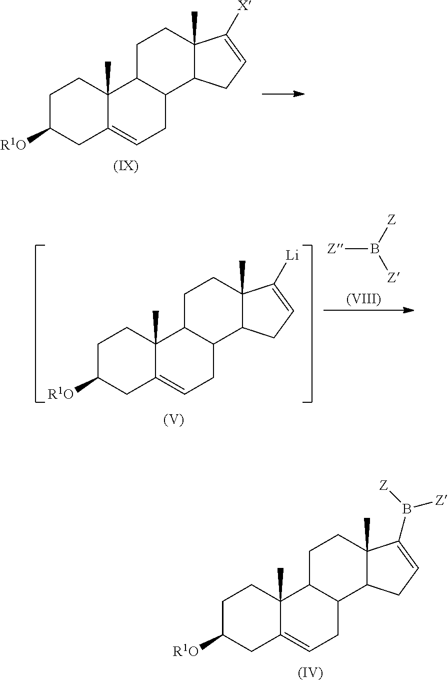

wherein
R¹, Z, Z′ and Z″ are as previously defined, and
X′ is bromo or iodo.

In a particular embodiment, X′ is iodo.

In a particular embodiment, R¹ is a silyl protecting group of formula Si(R³)(R⁴)(R⁵). Preferably, R³, R⁴ and R⁵ are independently selected from C₁-C₄ alkyl and C₆-C₁₄ aryl; more preferably are independently selected from methyl, i-propyl, t-butyl and phenyl. In a further embodiment, R¹ is trimethylsilyl (TMS), dimethylphenylsilyl (DMPS) or dimethyl-t-butylsilyl (TBDMS). Preferably, R¹ is TBDMS.

In a particular embodiment, Z, Z′ and Z″ are OH, methoxy, ethoxy or i-propoxy, or Z and Z′ form together an ethylendioxy, tetramethylethylenedioxy, propylendioxy, dimethylpropylendioxy, trimethylpropylendioxy, tetramethylpropylendioxy or benzene-1,2-dioxy group and Z″ is selected from OH, methoxy, ethoxy or i-propoxy. Preferably, Z, Z′ and Z″ are OH, methoxy or ethoxy. More preferably, Z, Z′ and Z″ are ethoxy.

The lithium base can be selected from alkyl lithium bases such as n-BuLi, sec-BuLi, tert-BuLi, Hexyl-Li. Preferably, the lithium base is n-BuLi or Hexyl-Li.

Suitable solvents for the preparation of a compound of formula (IV) include organic solvents, such as acyclic or cyclic ethers (e.g. Et₂O, iPr₂O, dioxane, tetrahydrofuran), hydrocarbon solvents (e.g. pentane, hexane), halogenated solvents (e.g. methylene chloride), aromatic solvents (e.g. toluene, xylene), or mixtures thereof. In a particular embodiment, the solvent is an acyclic or cyclic ether, preferably THF.

The compound of formula (IV) obtained according to the above methods can be directly used in the coupling reaction with a compound of formula (III) or can be previously transformed into a different compound of formula (IV). In a particular embodiment, a compound of formula (IV) wherein Z and Z′ are OH is obtained by reacting a compound of formula (V) with a compound of formula (VIII) wherein Z, Z′ and Z″ are selected from C₁-C₈ alkoxide, preferably methoxy, ethoxy or i-propoxy, followed by subsequent hydrolysis of the resulting boronic ester into the boronic acid. Suitable conditions for the hydrolysis of boronic esters into the boronic acids are well known in the art. In a particular embodiment said reaction is performed under acid conditions.

In a particular embodiment, a compound of formula (IV) wherein R¹ is TBDMS and Z and Z′ are ethoxy is obtained by reacting a compound of formula (IX) wherein R¹ is TBDMS and X′ is I with n-BuLi and a compound of formula (VIII) wherein Z, Z′ and Z″ are ethoxy, preferably in the presence of THF. This compound can be further hydrolyzed to give a compound of formula (IV) wherein R¹ is TBDMS and Z and Z′ are OH.

### Synthesis of Intermediate Compounds of Formula (IX)

Compounds of formula (IX) as defined above can be obtained by methods known in the art (e.g. as disclosed in EP0721461).

In a particular embodiment, compounds of formula (IX) are obtained by reacting a compound of formula (IIb) with a bromide or iodide source in the presence of a base:

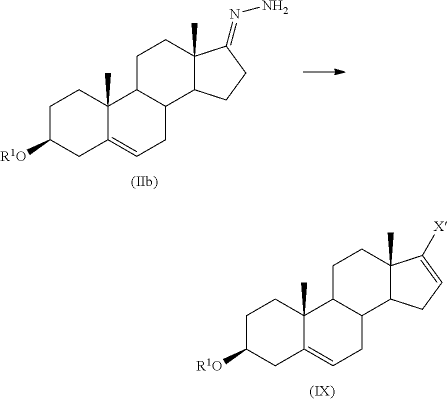

wherein R¹ and X′ are as previously defined.

In a particular embodiment, R¹ is selected from H and a silyl protecting group of formula Si(R³)(R⁴)(R⁵) wherein R³, R⁴ and R⁵ are independently selected from C₁-C₄ alkyl and C₆-C₁₄ aryl; more preferably are independently selected from methyl, i-propyl, t-butyl and phenyl. In a further embodiment, R¹ is selected from H, trimethylsilyl (TMS), dimethylphenylsilyl (DMPS) and dimethyl-t-butylsilyl (TBDMS). Preferably, R¹ is H or TBDMS, more preferably R¹ is H.

In a preferred embodiment, X′ is I.

Compounds of formula (IX) wherein X′ is Br can be obtained by using Br₂ or N-bromosuccinimide (NBS) as the bromide source.

Compounds of formula (IX) wherein X′ is I can be obtained by using I₂ or N-iodosuccinimide (NIS) as the iodide source.

Suitable basis for this reaction include non-nucleophilic bases, preferably non-nucleophilic organic bases such as non-nucleophilic amines, amidines or guanidine bases. Examples of suitable bases include, for example, triethylamine, diisopropylethylamine (DIPEA), 1,8-diazabicyclo[5.4.0]undec-7-ene (DBU), 1,5-diazabicyclo[4.3.0]non-5-ene (DBN), 1,1,3,3-tetramethylguanidine (TMG), triazabicyclodecene (TBD) or dicyclohexylguanidines. In a particular embodiment, the base is 1,1,3,3-tetramethylguanidine (TMG).

Suitable solvents for the preparation of a compound of formula (IX) include organic solvents, such as acyclic or cyclic ethers (e.g. Et₂O, iPr₂O, dioxane, tetrahydrofuran), hydrocarbon solvents (e.g. pentane, hexane), halogenated solvents (e.g. methylene chloride), aromatic solvents (e.g. toluene, xylene), or mixtures thereof. In a particular embodiment, the solvent is an acyclic or cyclic ether, preferably THF.

In a particular embodiment, a compound of formula (IX) wherein R¹ is H and X′ is I is obtained by reacting a compound of formula (IIb) wherein R¹ is H with I₂ and TMG, preferably, in the presence of THF. Said compound can be further protected to give a compound of formula (IX) wherein R¹ is TBDMS and X′ is I.

In a particular embodiment, compounds of formula (IIa) and (IIb) can be obtained by hydrazination of a compound of formula (VI)

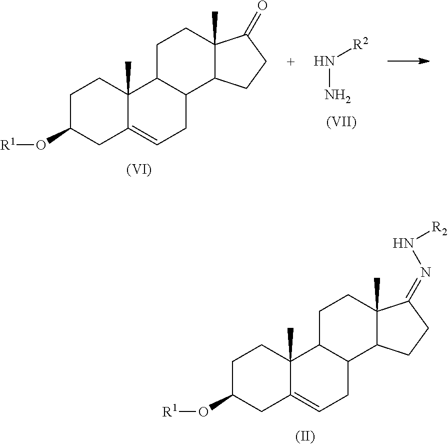

wherein:
R¹ is as previously defined, and
R² is selected from H and SO₂Ar, wherein Ar is an optionally substituted C₆-C₁₄ aryl. Preferably Ar is phenyl, tolyl, 2,4,6-trimethylphenyl or 2,4,6-triisopropylphenyl.

In a particular embodiment, compound of formula (VII) is hydrazine or a solvate thereof. In a further embodiment, compound of formula (VII) is hydrazine hydrate or hydrazine sulfate.

In a particular embodiment, R¹ is selected from H and a silyl protecting group of formula Si(R³)(R⁴)(R⁵) wherein R³, R⁴ and R⁵ are independently selected from C₁-C₄ alkyl and C₆-C₁₄ aryl; more preferably are independently selected from methyl, i-propyl, t-butyl and phenyl. In a further embodiment, R¹ is selected from H, trimethylsilyl (TMS), dimethylphenylsilyl (DMPS) and dimethyl-t-butylsilyl (TBDMS). Preferably, R¹ is H or TBDMS; more preferably R¹ is H.

The reaction can be catalyzed by using a suitable acid, such as p-TsOH or hydrazine sulfate.

It has been found that the condensation of a ketone of formula (VI) and a hydrazine of formula (VII), depending on the nature of R¹, may proceed with partial hydrolysis of the hydroxyl protecting group on position 3. Accordingly, in a particular embodiment, said reaction is initially carried out using a compound of formula (VI) wherein R¹ is H to obtain a compound of formula (IIa) or (IIb) wherein R¹ is H, which may be subsequently protected in a further step of the synthesis through common processes known in the state of the art.

In a particular embodiment, a compound of formula (II) wherein R¹ and R² are H is obtained by reacting a compound of formula (VI) wherein R¹ is H with hydrazine hydrate, preferably, in the presence of hydrazine sulfate and water. Said compound can be further protected to give a compound of formula (IIb) wherein R¹ is TBDMS.

The compounds of formula (IIa) and (IIb) thus obtained can be optionally isolated by precipitation through addition over water or by solvent evaporation and further precipitation through addition of an organic solvent such as diethyl ether, toluene, heptane or any other suitable solvent, depending on the nature of R¹.

Protection and deprotection of the hydroxyl group at position 3 (—OR¹) can be performed at any stage of the synthesis. The most suitable stage for said protection and/or deprotection can be readily determined by those skilled in the art. In a particular embodiment, the hydroxyl group at position 3 is protected in a compound of formula (IX) and is deprotected in a compound of formula (I), after the coupling reaction.

### Intermediate Compounds of the Processes

In a further aspect, the invention is directed to the compounds useful as intermediates in the process of the invention.

In an aspect, the invention is directed to a compound of formula (II)

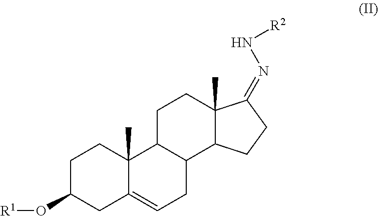

or a salt or solvate thereof, wherein
R¹ is selected from the group consisting of H and a hydroxyl protecting group (HPG);
R² is SO₂R⁷;
R⁷ is selected from the group consisting of optionally substituted C₁-C₈ alkyl and optionally substituted C₆-C₁₄ aryl;
with the proviso that the following compounds are not included:

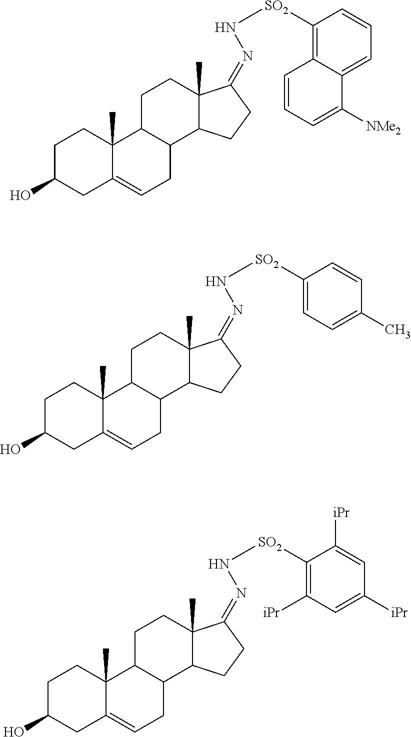

In a particular embodiment, R¹ is a silyl protecting group of formula Si(R³)(R⁴)(R⁵). Preferably, R³, R⁴ and R⁵ are independently selected from C₁-C₄ alkyl and C₆-C₁₄ aryl; more preferably are independently selected from methyl, i-propyl, t-butyl and phenyl. In a further embodiment, R¹ is trimethylsilyl (TMS), dimethylphenylsilyl (DMPS) or dimethyl-t-butylsilyl (TBDMS). Preferably, R¹ is TBDMS.

According to a particular embodiment, in the compound of formula (II) R¹ is selected from the group consisting of H, COMe, SitBuMe₂ (TBDMS) and SiMe₂Ph (DMPS). More preferably, R¹ is TBDMS.

According to another particular embodiment, in the compound of formula (II) R² is SO₂R⁷ wherein R⁷ is an optionally substituted C₆-C₁₄ aryl, such as Ph, Tol, 2,4,6-trimethylphenyl or 2,4,6-triisopropylphenyl.

In an aspect, the invention is directed to a compound of formula (IX)

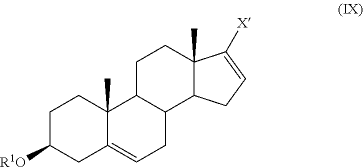

or a salt or solvate thereof, wherein
R¹ is a hydroxyl protecting group, and
X′ is bromo or iodo.

In a particular embodiment, X′ is iodo.

In a particular embodiment, R¹ is a silyl protecting group of formula Si(R³)(R⁴)(R⁵). Preferably, R³, R⁴ and R⁵ are independently selected from C₁-C₄ alkyl and C₆-C₁₄ aryl; more preferably are independently selected from methyl, i-propyl, t-butyl and phenyl. In a further embodiment, R¹ is trimethylsilyl (TMS), dimethylphenylsilyl (DMPS) or dimethyl-t-butylsilyl (TBDMS). Preferably, R¹ is TBDMS.

In a particular embodiment, X′ is I and R¹ is TBDMS.

In another aspect, the invention is directed to a compound of formula (V)

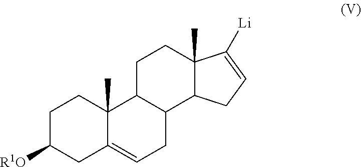

or a salt or solvate thereof, wherein
R¹ is a hydroxyl protecting group.

In a particular embodiment, R¹ is a silyl protecting group of formula Si(R³)(R⁴)(R⁵). Preferably, R³, R⁴ and R⁵ are independently selected from C₁-C₄ alkyl and C₆-C₁₄ aryl; more preferably are independently selected from methyl, i-propyl, t-butyl and phenyl. In a further embodiment, R¹ is trimethylsilyl (TMS), dimethylphenylsilyl (DMPS) or dimethyl-t-butylsilyl (TBDMS). Preferably, R¹ is TBDMS.

In another aspect, the invention is directed to a compound of formula (IV)

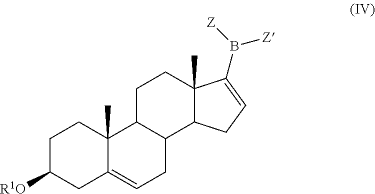

or a salt or solvate thereof, wherein
R¹ is selected from the group consisting of H and a hydroxyl protecting group (HPG); and
Z and Z′ are independently selected from the group consisting of hydroxyl, optionally substituted C₁-C₈ alkoxy and optionally substituted C₁-C₈ alkyl, or Z and Z′ together form an optionally substituted C₂-C₃ alkylenedioxy group or an optionally substituted C₆ aryldioxy.

In a particular embodiment, R¹ is a silyl protecting group of formula Si(R³)(R⁴)(R⁵). Preferably, R³, R⁴ and R⁵ are independently selected from C₁-C₄ alkyl and C₆-C₁₄ aryl; more preferably are independently selected from methyl, i-propyl, t-butyl and phenyl. In a further embodiment, R¹ is trimethylsilyl (TMS), dimethylphenylsilyl (DMPS) or dimethyl-t-butylsilyl (TBDMS). Preferably, R¹ is TBDMS.

In a particular embodiment, Z and Z′ are OH, methoxy, ethoxy or i-propoxy, or Z and Z′ form together an ethylendioxy, tetramethylethylenedioxy, propylendioxy, dimethylpropylendioxy, trimethylpropylendioxy, tetramethylpropylendioxy or benzene-1,2-dioxy group. Preferably, Z and Z′ are OH, methoxy or ethoxy. More preferably, Z and Z′ are OH or ethoxy.

In a particular embodiment, R¹ is TBDMS and Z and Z′ are ethoxy.

In another embodiment, R¹ is TBDMS and Z and Z′ are OH.

In a further aspect, the present invention refers to compounds of formula (I), wherein R¹ is SiR³R⁴R⁵, or a salt or solvate thereof. 3-((Tert-butyldimethylsilyl)oxy)abiraterone (3-TBDMS-abiraterone) and its chlorhydrate salt represent preferred compounds of the invention.

Salts of silyl ethers of a compound of formula (I) may be recovered from a solution of the free base in any suitable solvent, or mixture of solvents, by treating the solution with the corresponding acid. Suitable solvents include esters and ethers. Esters which may be used include esters withy acetic acid, such as methyl acetate, ethyl acetate and isopropyl acetate. Ethers which may be used include diethyl ether, diisopropyl ether, methyl tert-butyl ether (MTBE), and especially tetrahydrofuran (THF), which gave a particularly good recovery of the salt. Preferably the acid is hydrochloric acid, such as HCl aq (1M).

According to a preferred embodiment, the chlorhydrate salt of 3-TBDMS-abiraterone is prepared by recovering the salt from a solution of the free base in any suitable solvent, more preferably THF, by treating the solution with hydrochloric acid.

In additional preferred embodiments, the preferences described above for the different substituents in the intermediates and compounds of the invention as well as for the conditions of the processes for their preparation are combined. The present invention is also directed to such combinations of preferred substitutions in the chemical formulae above and conditions of the processes for obtaining the same.

The following examples are merely illustrative of certain embodiments of the invention and cannot be considered as restricting it in any way.

### EXAMPLES

### Example 1

### Synthesis of 5-Dehydroepiandrosterone-17-Hydrazone

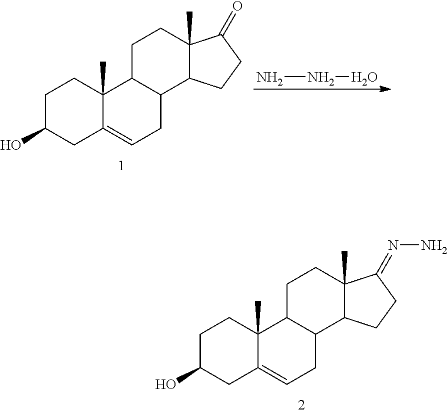

Hydrazine monohydrate (9.75 ml, 200 mmol) and a solution of hydrazine sulfate (0.0325 g, 0.25 mmol) in water (1 mL) was added to a suspension of 5-DHEA 1 (14.4 g, 49.93 mmol) in ethanol (250 mL). The mixture was stirred at room temperature for about three days and was followed by TLC. The reaction mixture was poured into water (1 L) and the resulting white precipitate was filtered and washed with water (3×30 ml) and ether (3×10 ml). The title compound was obtained as a crystalline solid (95% yield).

### Example 2

### Synthesis of 17-iodo-5,16-androstadien-3-ol

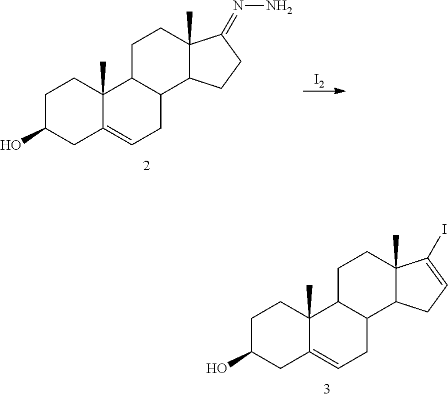

A solution of compound 2 (14 g, 46.28 mmol) in THF (350 mL) was slowly added (for about 1 hour) through an addition funnel to an ice-cold solution of I₂ (24.67 g, 97.19 mmol) and 1,1,3,3-tetramethylguanidine (29 ml, 231.4 mmol) in THF (920 mL). When the reaction was complete, the mixture was filtered and the filtrate was concentrated under vacuum to yield a brown oil. The oil was dissolved in ether, washed with HCl 1M until the aqueous phase was acidic and then sequentially washed with NaOH 0.5 M, Na₂S₂O₃ 1M and water. The organic phase was separated, dried over MgSO₄ and concentrated under vacuum to yield a product which was crystallized from ether/heptane (90% yield).

### Example 3

### Synthesis of 3-((tert-butyldimethylsilyl)oxy)-17-iodo-5,16-androstadiene

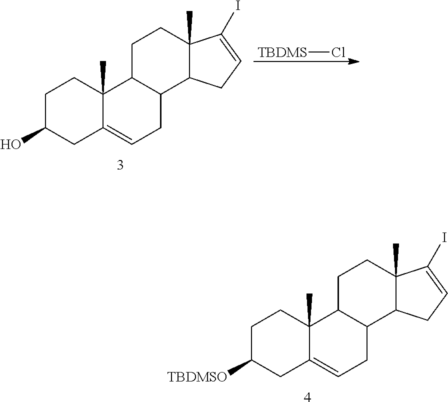

Imidazol (2.87 g, 42.14 mmol) was added to a suspension of compound 3 (3.5 g, 8.78 mmol) in methylene chloride (30 ml). The mixture was stirred for 10 minutes until complete dissolution of the reagents. TBDMSCl was then added (1.85 g, 12.30 mmol). The reaction mixture was stirred at room temperature for 1 h and followed by TLC. The solvent was evaporated under vacuum yielding a white precipitate which was washed with HCl 1M (2×20 ml) and then with water (90% yield).

¹H-NMR (400 MHz, CDCl₃): 6.14 (dd, J=3.2, 1.7 Hz, 1H), 5.32 (d, J=5.3 Hz, 1H), 3.48 (s, 1H), 1.03 (s, 3H), 0.89 (s, 9H), 0.75 (s, 3H), 0.06 (s, 6H).

¹³C-NMR (400 MHz, CDCl₃): 141.9, 137.5, 120.6, 112.7, 72.5, 54.8, 50.5, 49.9, 42.7, 37.2, 36.8, 36.1, 33.7, 32.0, 31.2, 31.0, 25.9, 20.8, 19.3, 18.3, 15.1, −4.6.

### Example 4

### Synthesis of 3-((tert-butyldimethylsilyl)oxy)-5,16-androstadien-17-boronic acid

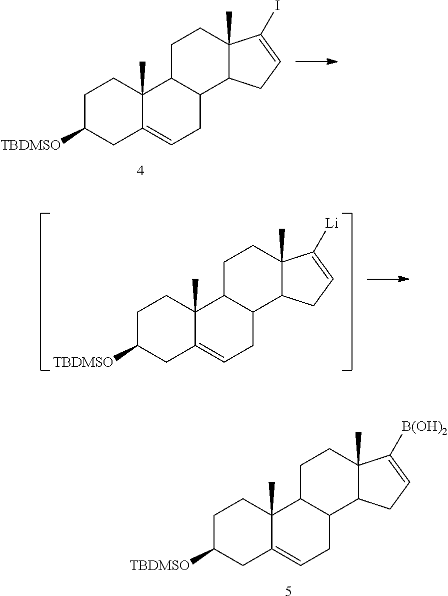

B(OEt)₃ (1.5 ml, 8.76 mmol) was added to a solution of compound 4 (1.5 g, 2.92 mmol) in dry THF (15 mL) at −78° C. After stirring for 10 minutes, n-BuLi (4.5 mL, 2M) was added at about −65° C. The mixture was stirred for 10 minutes and then poured over water (100 mL). The reaction mixture was extracted with ethyl acetate. The organic phase was dried over MgSO₄, filtered and evaporated. The resulting residue was washed with heptanes (3×10 ml) to yield a white solid (90% yield).

¹H-NMR (500 MHz, CDCl₃): 6.86 (d, J=7.2 Hz, 1H), 5.34 (d, J=4.0 Hz, 1H), 3.49 (s, 1H), 1.06 (d, J=3.2 Hz, 3H), 0.89 (s, 9H), 0.86 (d, J=7.1 Hz, 3H), 0.06 (s, 6H).

¹³C-NMR (500 MHz, CDCl₃): 150.8, 141.9, 120.9, 72.6, 57.1, 50.8, 47.2, 47.1, 42.9, 37.3, 36.8, 33.6, 32.1, 32.0, 30.7, 25.9, 25.6, 25.2, 21.0, 19.4, 19.3, 18.2, 16.8, 16.7, 13.9, −4.6.

### Example 5

### Synthesis of 3-((tert-butyldimethylsilyl)oxy)abiraterone

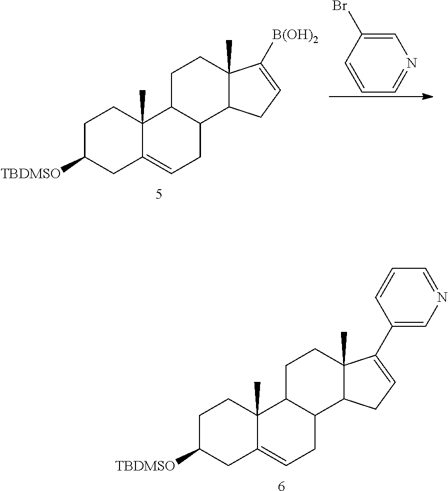

A solution of Na₂CO₃ (0.079 g, 0.75 mmol) in water (0.4 mL) under inert atmosphere was prepared. Compound 5 (0.215 g, 0.5 mmol) and dry THF (2 mL) were added. Toluene (3 mL) was added to the resulting solution giving rise to the formation of a white precipitate. Solvents were deoxygenates and Pd(PPh₃)₄ (6%, 0.035 g, 0.03 mmol) was added. Finally, 3-bromopyridine (1.2 eq, 0.06 ml, 0.6 mmol) was added. The reaction mixture was refluxed overnight. Volatiles were evaporated under vacuum, HCl 6M (10 mL) was added and the resulting white solid was filtered and washed with water. A solution of NaHCO₃ 1M (20 mL) was added and the mixture was extrated with ethyl acetate. The organic phase was dried over MgSO₄, filtered and dried to afford a white solid (83% yield).

### Example 6

### Synthesis of 3-((tert-butyldimethylsilyl)oxy)abiraterone

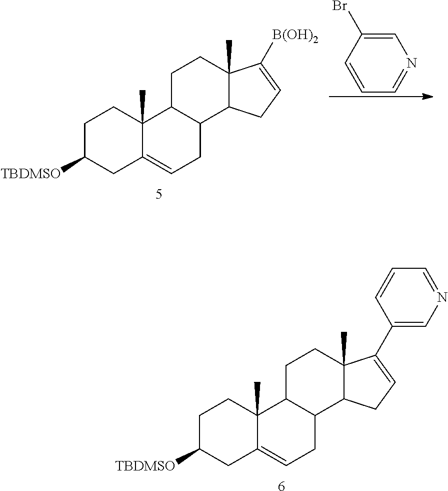

A solution of K₂CO₃ (0.917 g) in water (4.5 mL) under inert atmosphere was prepared. Compound 5 (2.5 g) and dry THF (28 mL) were added, giving rise to the formation of a white precipitate. Solvents were deoxygenates and dichloro [1,1′-bis(diphenylphosphino)ferrocene] palladium(II)dichloromethane adduct (0.232 g) was added. Finally, 3-bromopyridine (0.7 ml) was added. The reaction mixture was refluxed overnight. The reaction mixture was cooled and filtered off. The liquid phase was washed with brine. Volatiles were evaporated under vacuum and isopropyl ether was added and evaporated under vacuum. HCl 6M (100 mL) was added and the resulting white solid was filtered, washed with water and dried to afford a white solid (72% yield).

### Example 7

### Synthesis of 3-((tert-butyldimethylsilyl)oxy)abiraterone

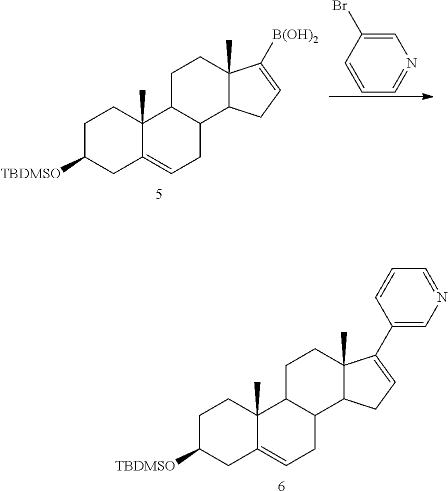

A solution of Na₂CO₃ (3.67 g) in water (18.5 mL) under inert atmosphere was prepared. Compound 5 (10.0 g, 23.2 mmol) and dry THF (100 mL) were added. Toluene (100 mL) and water (18.6 mL) were added giving rise to the formation of a white precipitate. Solvents were deoxygenates and dichloro [1,1′-bis(diphenylphosphino)ferrocene] palladium(II)dichloromethane adduct (0.140 g) was added. Finally, 3-bromopyridine (2.8 ml) was added. The reaction mixture was refluxed overnight. The reaction mixture was cooled and filtered off. The liquid phase was washed with brine. Volatiles were evaporated under vacuum and isopropyl ether was added and evaporated under vacuum. HCl 1M (100 mL) was added and the resulting white solid was filtered, washed with water and dried to afford a white solid (77% yield).

### Example 8

### Synthesis of abiraterone from 3-((tert-butyldimethylsilyl)oxy)abiraterone

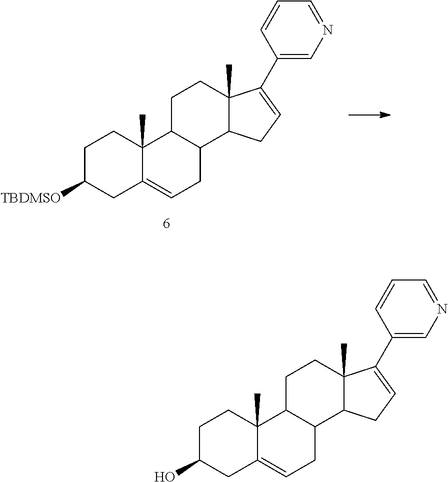

In a round bottom flask were dissolved 0.463 g (1 mmol) of 3-((tert-butyldimethylsilyl)oxy)abiraterone in 10 ml of THF and 7.5 ml of a 1 M solution of tetrabutylammonium fluoride in THF was added. The reaction was monitored by TLC until complete deprotection of the hydroxyl group. Once the reaction was complete, the solvent was evaporated partially under reduced pressure and some water was added to the reaction mixture to afford a suspension. The solid formed was isolated by filtration, washed with water (3×5 ml) and dried under vacuum to afford 0.31 g (89% yield) of pure abiraterone, as confirmed by NMR.

### Example 9

### Synthesis of 5-dehydroepiandrosterone p-toluenesulfonyl hydrazone

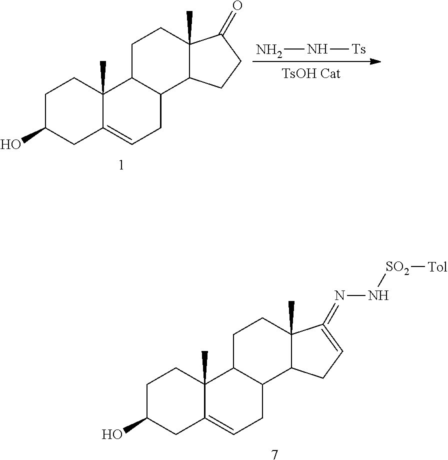

In a round bottom flask under inert atmosphere were added consecutively 10 g (34.67 mmol) of 5-DHEA 1, 7.32 g of NH₂NHTs (38.13 mmol), 0.66 g (10%) of p-toluensulfonic acid and 70 ml of THF. The solution obtained was heated to boiling point for about 2-3 hours until the reaction was complete by TLC. The reaction mixture was cooled and the solvent evaporated under reduced pressure to afford a solid residue, which was resuspended in ethyl ether and filtered to yield 15.6 g of the corresponding 5-dehydroepiandrosterone p-toluenesulfonyl hydrazone 7 (98% yield). Characteristic NMR signals:

¹H NMR (400 MHz, CDCl₃) δ 7.82 (d, J=8.3 Hz, 2H), 7.29 (d, J=8.1 Hz, 2H), 5.33 (d, J=5.1 Hz, 1H), 3.51 (s, 1H), 2.42 (s, 3H), 1.00 (s, 3H), 0.82 (s, 3H).

### Example 10

### Synthesis of 5-dehydroepiandrosterone-2,4,6-trimethylbenzenesulfonyl hydrazone

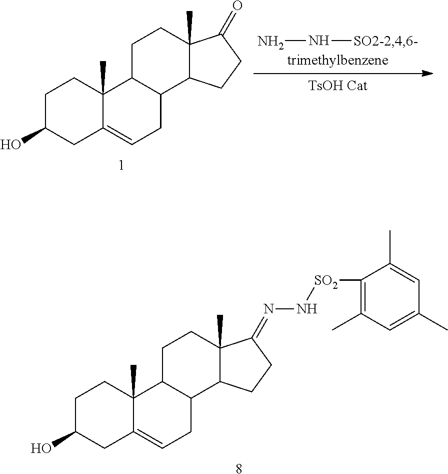

In a round bottom flask under inert atmosphere were added consecutively 2 g (6.93 mmol) of 5-DHEA 1, 1.63 g of 2,4,6-trimethylbenzenesulfonyl hydrazine (7.62 mmol), 0.133 g (10%) of p-toluensulfonic acid and 30 ml of THF. The solution obtained was heated to boiling point for about 2-3 hours until the reaction was complete by TLC. The reaction mixture was cooled and the solvent evaporated under reduced pressure to afford a solid residue, which was resuspended in methanol and filtered to yield 3 g of the corresponding 5-dehydroepiandrosterone-2,4,6-trimethylbenzenesulfonyl hydrazone 8 (89% yield). Characteristic NMR signals:

¹H NMR (400 MHz, CDCl₃) δ 7.02 (s, 1H), 6.94 (s, 2H), 5.33 (d, J=5.2 Hz, 1H), 3.51 (5, 1H), 2.66 (s, 6H), 2.29 (s, 3H), 1.00 (s, 3H), 0.76 (s, 3H).

### Example 11

### Synthesis of 5-dehydroepiandrosterone benzenesulfonyl hydrazone

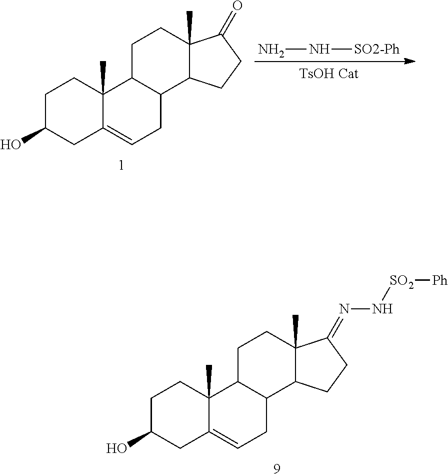

In a round bottom flask under inert atmosphere were added consecutively 2 g (6.93 mmol) of 5-DHEA 1, 1.43 g of NH₂NHSO₂-Ph (8.31 mmol), 0.133 g (10%) of p-toluensulfonic acid and 30 ml of THF. The solution obtained was heated to boiling point for about 2-3 hours until the reaction was complete by TLC. The reaction mixture was cooled and the solvent evaporated under reduced pressure to afford a solid residue, which was resuspended in ether and filtered to yield 2.75 g of the corresponding 5-dehydroepiandrosterone benzenesulfonyl hydrazone 9 (89% yield).

### Example 12

### Synthesis of 5-dehydroepiandrosterone 2,4,6-tri-isopropylbenzenesulfonyl hydrazone

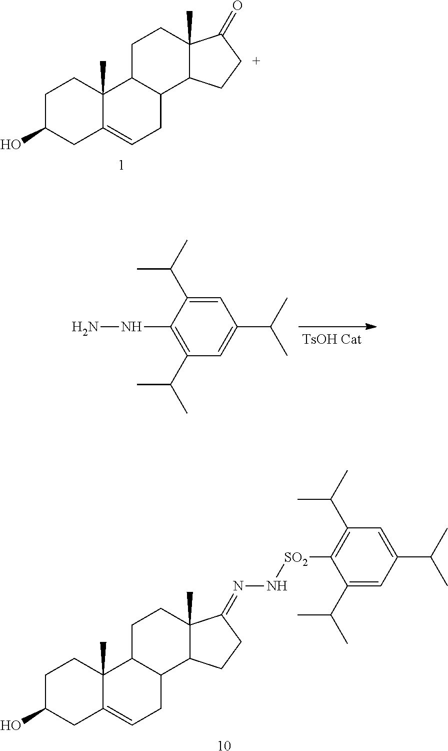

In a round bottom flask under inert atmosphere were added consecutively 0.4 g (1.39 mmol) of 5-DHEA 1, 0.45 g of 2,4,6-tri-isopropylbenzenesulfonyl hydrazine (1.52 mmol), 0.026 g (10%) of p-toluensulfonic acid and 5 ml of THF. The solution obtained was heated to boiling point for about 2-3 hours until the reaction was complete by TLC. The reaction mixture was cooled and the solvent evaporated under reduced pressure to afford a solid residue, which was resuspended in heptane and filtered. The mother liquors were partially concentrated and cooled, affording a precipitate which was filtered again. The solids combined and dried yield 0.53 g of the corresponding dehydroepiandrosterone 2,4,6-tri-isopropylbenzenesulfonyl hydrazone 10 (67% yield).

### Example 13

### Synthesis of 3-acetyl-5-dehydroepiandrosterone benzenesulfonyl hydrazone

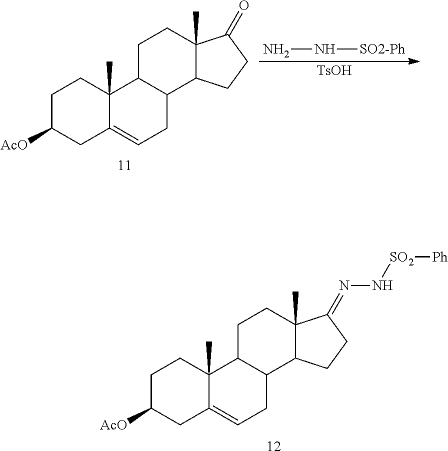

In a round bottom flask under inert atmosphere were added consecutively 15 g (45.39 mmol) of 5-DHEA-acetate 11, 9.38 g of NH₂NHSO₂-Ph (54 mmol), 0.86 g (10%) of p-toluensulfonic acid and 60 ml of THF. The solution obtained was heated to boiling point for about 2-3 hours until the reaction was complete by TLC. The reaction mixture was cooled and the solvent was evaporated under reduced pressure to afford a solid residue, which was resuspended in ether and filtered to yield 20.80 g of the corresponding 3-acetyl-5-dehydroepiandrosterone benzenesulfonyl hydrazone 12 (94% yield), along with a 10% of the alcohol compound as an impurity, resulting from a partial hydrolysis of the acetate group under the reaction conditions. Characteristic NMR signals:

¹H NMR (400 MHz, CDCl₃) δ 7.95 (dd, J=8.4, 1.2 Hz, 2H), 7.58 (d, J=7.5 Hz, 1H), 7.55-7.43 (m, 2H), 7.15 (s, 1H), 5.35 (d, J=5.0 Hz, 1H), 4.66-4.51 (m, 1H), 3.47 (d, J=31.6 Hz, 1H), 2.03 (s, 3H), 1.02 (s, 3H), 0.79 (s, 3H).

### Example 14

### Synthesis of 3-((tert-butyldimethylsilyl)oxy)-5-dehydroepiandrosterone p-toluenesulfonyl hydrazone

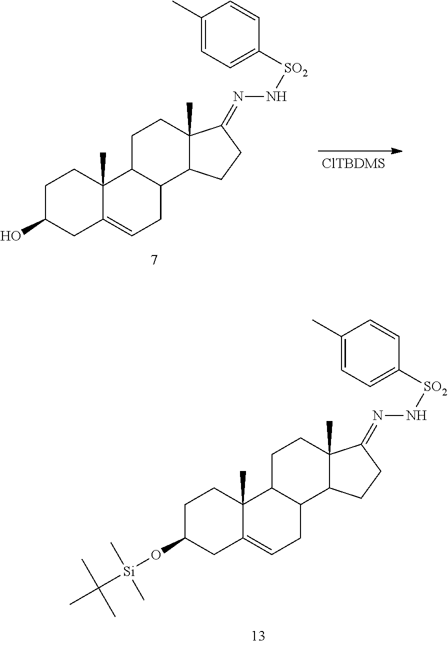 [SMILES: Cc1ccc(S(=O)(=O)N/N=C2\CCC3C4CC=C5C[C@@H](O)CC[C@]5(C)C4CC[C@]23C)cc1 ; Cc1ccc(S(=O)(=O)N/N=C2\CCC3C4CC=C5C[C@@H](O[Si](C)(C)C(C)(C)C)CC[C@]5(C)C4CC[C@]23C)cc1 ; "ClTBDMS"]

Over a suspension of 10 g (21.90 mmol) of 5-dehydroepiandrosterone p-toluenesulfonyl hydrazone 7 and 50 ml of CH₂Cl₂ in a round bottom flask under inert atmosphere were added 7.15 g (4.8 eq) of imidazol. The reaction mixture was stirred at room temperature until a yellowish solution was obtained (about 10 min). Over the solution obtained 4.42 g (1.3 eq) of TBDMSiCl were added, maintaining stirring for 2 hours more until the reaction was complete by TLC.

The solvent was evaporated under reduced pressure to afford an oily residue. 20 ml of acetone and 100 ml of water were consecutively added and the mixture was stirred, affording a white solid as precipitate.

100 ml of water were added again over the suspension and it was filtered. The solid obtained was washed with water and dried under reduced pressure to yield 10.93 g of the corresponding 3-((tert-butyldimethylsilyl)oxy)-5-dehydroepiandrosterone p-toluenesulfonyl hydrazone 13 (88% yield). Characteristic NMR signals:

¹H NMR (400 MHz, CDCl₃): δ 7.83 (d, J=8.3 Hz, 2H), 7.29 (d, J=8.1 Hz, 2H), 5.29 (d, J=5.0 Hz, 1H), 3.46 (d, J=4.7 Hz, 1H), 2.42 (s, 3H), 1.00 (s, 3H), 0.88 (s, 9H), 0.78 (s, 3H), 0.05 (s, 6H).

### Example 15

### Synthesis of 3-((tert-butyldimethylsilyl)oxy)-5-dehydroepiandrosterone 2,4,6-trimethylbenzenesulfonyl hydrazone

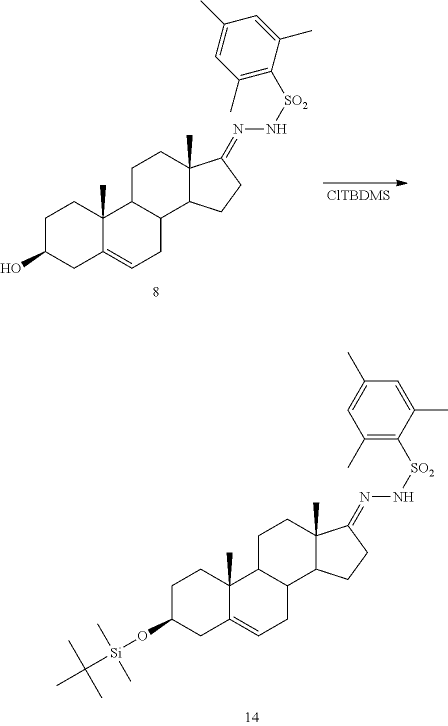

Over a suspension of 1 g (2.06 mmol) of 5-dehydroepiandrosterone-2,4,6-trimethylbenzenesulfonyl hydrazone 8 and 20 ml of CH₂Cl₂ in a round bottom flask under inert atmosphere were added 0.67 g (4.8 eq) of imidazol. The reaction mixture was stirred at room temperature until a yellowish solution was obtained (about 10 min). Over the solution obtained 0.62 g (2 eq) of TBDMSiCl were added, maintaining stirring for 3 hours more until the reaction was complete by TLC.

The solvent was evaporated under reduced pressure to afford a solid residue, which was resuspended in water. Then, it was filtered, washed with water and dried under reduced pressure to yield 1.19 g of the corresponding 3-((tert-butyldimethylsilyl)oxy)-5-dehydroepiandrosterone 2,4,6-trimethylbenzenesulfonyl hydrazone 14 (96% yield). Characteristic NMR signals:

¹H NMR (400 MHz, CDCl₃): δ 6.94 (d, J=4.0 Hz, 3H), 5.30 (s, 1H), 3.53-3.40 (m, 1H), 2.67 (s, 6H), 2.30 (s, 3H), 0.99 (s, 3H), 0.88 (s, 9H), 0.75 (s, 3H), 0.05 (s, 6H).

### Example 16

### Synthesis of 3-((tert-butyldimethylsilyl)oxy)-5-dehydroepiandrosterone benzenesulfonyl hydrazone

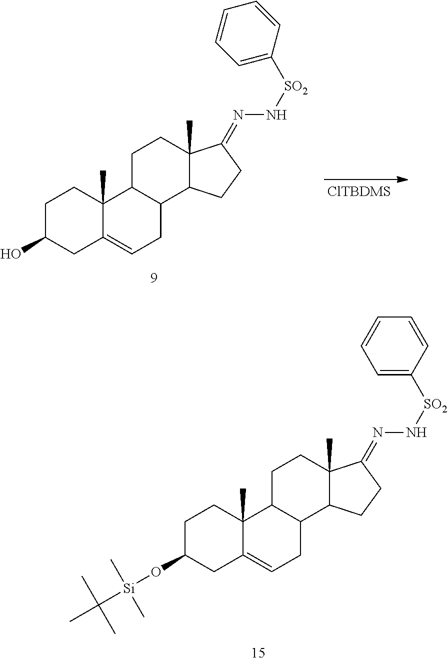 [SMILES: C[C@]12CC[C@H](O)CC1=CCC1C2CC[C@]2(C)/C(=N/NS(=O)(=O)c3ccccc3)CCC12 ; CC(C)(C)[Si](C)(C)O[C@H]1CC[C@@]2(C)C(=CCC3C2CC[C@]2(C)/C(=N/NS(=O)(=O)c4ccccc4)CCC32)C1 ; "ClTBDMS"]

Over a suspension of 4 g (9.04 mmol) of 5-dehydroepiandrosterone benzenesulfonyl hydrazone 9 and 50 ml of CH₂Cl₂ in a round bottom flask under inert atmosphere were added 2.95 g (4.8 eq) of imidazol. The reaction mixture was stirred at room temperature until a yellowish solution was obtained (about 10 min). Over the solution obtained 2.11 g (1.5 eq) of TBDMSiCl were added, maintaining stirring for 3 hours more until the reaction was complete by TLC.

The solvent was evaporated under reduced pressure to afford an oily residue. 8 ml of acetone and 50 ml of water were consecutively added and the mixture was stirred, affording a white solid as precipitate.

The suspension was filtered and the solid obtained was washed with water and dried under reduced pressure to yield 4.79 g of the corresponding 3-((tert-butyldimethylsilyl)oxy)-5-dehydroepiandrosterone benzenesulfonyl hydrazone 15 (93% yield). Characteristic NMR signals:

¹H NMR (400 MHz, CDCl₃): δ (dd, J=8.4, 1.3 Hz, 2H), 7.65-7.41 (m, 3H), 7.14 (s, 1H), 5.29 (d, J=5.1 Hz, 1H), 3.46 (s, 1H), 1.00 (s, 3H), 0.90-0.87 (m, 9H), 0.78 (s, 3H), 0.05 (s, 7H).

### Example 17

### Synthesis of 3-((tert-butyldimethylsilyl)oxy)-5-dehydroepiandrosterone 2,4,6-tri-isopropylbenzenesulfonyl hydrazone

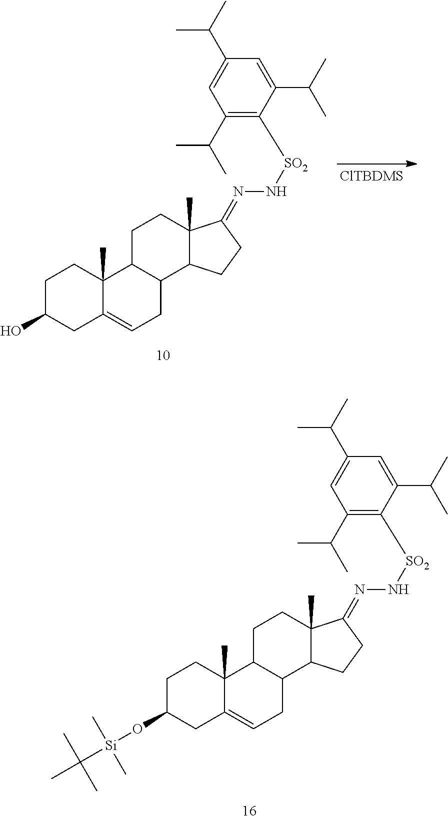

Over a suspension of 0.5 g (0.88 mmol) of 5-dehydroepiandrosterone-2,4,6-tri-isopropyllbenzenesulfonyl hydrazone 10 and 15 ml of CH₂Cl₂ in a round bottom flask under inert atmosphere were added 0.29 g (4.8 eq) of imidazol. The reaction mixture was stirred at room temperature until a yellowish solution was obtained (about 10 min). Over the solution obtained 0.2 g (1.5 eq) of TBDMSiCl were added, maintaining stirring for 3 hours more until the reaction was complete by TLC.

The solvent was evaporated under reduced pressure to afford an oily residue. 1 ml of acetone and 25 ml of water were consecutively added and the mixture was stirred, affording a white solid as precipitate.

The suspension was filtered and the solid obtained was washed with water and dried under reduced pressure to yield 0.5 g of the corresponding 3-((tert-butyldimethylsilyl)oxy)-5-dehydroepiandrosterone 2,4,6-triisopropylbenzenesulfonyl hydrazone 16 (73% yield). Characteristic NMR signals:

¹H NMR (400 MHz, CDCl₃): δ 7.15 (s, 2H), 6.94 (s, 1H), 5.48-5.42 (m, 1H), 5.30 (d, J=5.1 Hz, 1H), 4.27-4.11 (m, 2H), 3.98-3.96 (m, 1H), 3.46 (s, 1H), 2.90 (s, 1H), 1.26 (t, J=6.1 Hz, 18H), 0.98 (s, 3H), 0.88 (s, 9H), 0.74 (s, 3H), 0.05 (s, 6H).

### Example 18

### Synthesis of 3-((tert-butyldimethylsilyl)oxy)-5-dehydroepiandrosterone

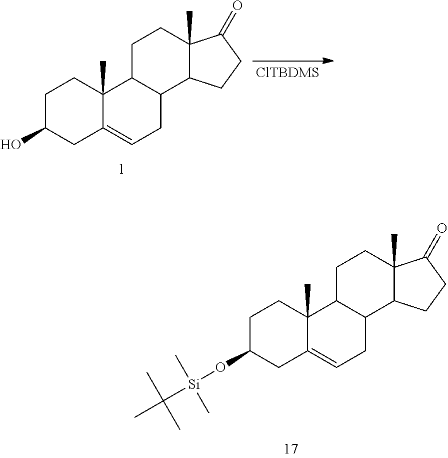 [SMILES: C[C@]12CC[C@H](O)CC1=CCC1C2CC[C@]2(C)C(=O)CCC12 ; CC(C)(C)[Si](C)(C)O[C@H]1CC[C@@]2(C)C(=CCC3C2CC[C@]2(C)C(=O)CCC32)C1 ; "ClTBDMS"]

Over a suspension of 2 g (6.93 mmol) of 5-dehydroepiandrosterone 1 and 20 ml of CH₂Cl₂ in a round bottom flask under inert atmosphere were added 2.26 g (4.8 eq) of imidazol. The reaction mixture was stirred at room temperature until a yellowish solution was obtained (about 10 min). Over the solution obtained 1.4 g (1.3 eq) of TBDMSiCl were added, maintaining stirring for 3 hours more until the reaction was complete by TLC.

The solvent was evaporated under reduced pressure to afford an oily residue. 5 ml of acetone and 40 ml of water were consecutively added and the mixture was stirred, affording a white solid as precipitate.

The suspension was filtered and the solid obtained was washed with water and dried under reduced pressure to yield 2.33 g of the corresponding 3-((tert-butyldimethylsilyl)oxy)-5-dehydroepiandrosterone 17 (83% yield). Characteristic NMR signals:

¹H NMR (400 MHz, CDCl₃): δ 5.34 (d, J=5.1 Hz, 1H), 3.48 (d, J=4.8 Hz, 1H), 1.02 (s, 3H), 0.88 (d, J=2.9 Hz, 12H), 0.06 (s, 6H).

### Example 19

### Synthesis of abiraterone

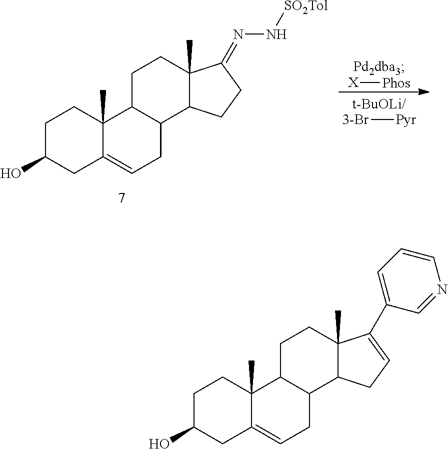

In a round bottom flask under inert atmosphere were added consecutively: 0.235 g (0.5 mmol) of 5-dehydroepiandrosterone p-toluenesulfonyl hydrazone 7, 0.0092 g (0.01 mmol, 2%) of Pd₂(dba)₃, 0.0095 g (0.02 mmol, 4%) of XPhos and 0.264 g (1.65 mmol, 3,3 eq) of ^{t}BuOLi. Thereafter 6 ml of dry dioxane and 0.067 ml (0.7 mmol, 1.4 eq) of 3-bromopyridine were also added. The suspension obtained was heated at reflux for 4 hours, and then it was cooled and filtered, washing the insoluble residue with THF (3×5 ml). The filtrates were combined and the solvent was evaporated under reduced pressure. In the residue obtained, abiraterone was detected as confirmed by NMR.

¹H NMR (400 MHz, CDCl₃): δ 8.62 (d, J=1.7 Hz, 1H), 8.46 (dd, J=4.8, 1.4 Hz, 1H), 7.64 (dd, J=5.9, 4.0 Hz, 1H), 7.24-7.19 (m, 1H), 6.00 (dd, J=3.2, 1.8 Hz, 1H), 5.46-5.31 (m, 1H), 3.54 (ddd, J=15.5, 11.0, 4.3 Hz, 1H), 1.07 (s, 3H), 1.05 (s, 3H), −0.00 (s, 2H).

### Example 20

### Synthesis of abiraterone acetate from 3-acetyl-5-dehydroepiandrosterone toluenesulfonyl hydrazone

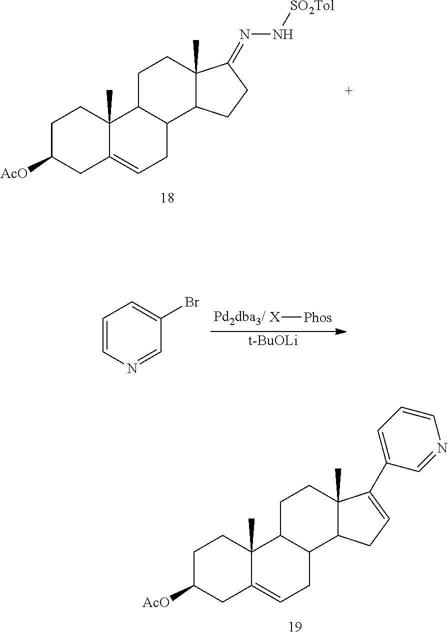

In a round bottom flask under inert atmosphere were added consecutively: 0.39 g (0.78 mmol) of 3-acetyl-5-dehydroepiandrosterone toluenesulfonyl hydrazone 18, 0.025 g (0.027 mmol, 3.4%) of Pd₂(dba)₃, 0.027 g (0.056 mmol, 7.18%) of XPhos. Thereafter 5 ml of dry dioxane were added to afford a suspension with stirring and 0.11 ml (1.17 mmol, 1.5 eq) of 3-bromopyridine and 0.41 g (5.15 mmol, 6.6 eq) of ^{t}BuOLi were consecutively also added to the suspension. The reaction mixture was heated at reflux for 4 hours, and then it was cooled and filtered, washing the insoluble residue with THF (3×5 ml). The filtrates were combined and the solvent was evaporated under reduced pressure to afford a residue wherein abiraterone acetate 19 was detected as one of the main products as confirmed by NMR.

### Example 21

### Synthesis of 3-((tert-butyldimethylsilyl)oxy)abiraterone from 3-((tert-butyldimethysilyl)oxy)-5-dehydroepiandrosterone p-toluenesulfonyl hydrazone

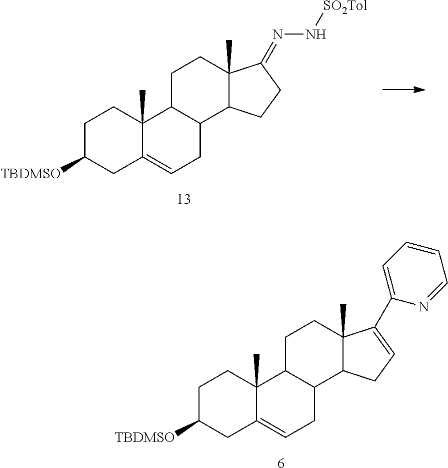

### a) General Reaction:

In a round bottom flask under inert atmosphere were added consecutively: 1 g (1.75 mmol) of 3-((tert-butyldimethylsilyl)oxy)-5-dehydroepiandrosterone p-toluenesulfonyl hydrazone 13, 0.032 g (0.035 mmol, 2%) of Pd₂(dba)₃, 0.033 g (0.069 mmol, 4%) of XPhos and 0.35 g (4.37 mmol, 2.5 eq) of t-BuOLi. Then, 30 ml of dry dioxane were added to form a suspension with stirring, over which 0.25 ml (2.62 mmol, 1.5 eq) of 3-bromopyridine were added.

The reaction mixture was heated at reflux for 4 hours, and then it was cooled and filtered, washing the insoluble residue with THF (3×5 ml). The filtrates were combined and the solvent was evaporated under reduced pressure to afford a residue containing the desired product (45%), starting material (5%) and a dimeric impurity resulting from two molecules of the starting material (24%).

### b) Using More Catalyst

In a round bottom flask under inert atmosphere were added consecutively: 0.57 g (1 mmol) of 3-((tert-butyldimethylsilyl)oxy)-5-dehydroepiandrosterone p-toluenesulfonyl hydrazone 13, 0.055 g (0.06 mmol, 6%) of Pd₂(dba)₃, 0.057 g (0.12 mmol, 12%) of XPhos and 0.2 g (2.5 mmol, 2.5 eq) of t-BuOLi. Then, 15 ml of dry dioxane were added to form a suspension with stirring, over which 0.14 ml (1.50 mmol, 1.5 eq) of 3-bromopyridine were added.

The reaction mixture was heated at 110° C. for 15 hours, and then it was cooled and filtered, washing the insoluble residue with THF (3×5 ml). The filtrates were combined and the solvent was evaporated under reduced pressure to afford a residue containing the desired product (43%) and a dimeric impurity resulting from two molecules of the starting material (8.5%).

### c) Using More Catalyst and Base

In a round bottom flask under inert atmosphere were added consecutively: 0.57 g (1 mmol) of 3-((tert-butyldimethylsilyl)oxy)-5-dehydroepiandrosterone p-toluenesulfonyl hydrazone 13, 0.055 g (0.06 mmol, 6%) of Pd₂(dba)₃, 0.057 g (0.12 mmol, 12%) of XPhos and 0.6 g (7.5 mmol, 7.5 eq) of t-BuOLi. Then, 15 ml of dry dioxane were added to form a suspension with stirring, over which 0.14 ml (1.50 mmol, 1.5 eq) of 3-bromopyridine were added.

The reaction mixture was heated at 110° C. for 15 hours, and then it was cooled and filtered, washing the insoluble residue with THF (3×5 ml). The filtrates were combined and the solvent was evaporated under reduced pressure to afford a residue containing the desired product (62%) and a dimeric impurity resulting from two molecules of the starting material (15.4%).

The residue was solved in 10 ml of THF and 17 ml of HCl aq (1M) were added and a suspension was obtained. The solid obtained was filtered at room temperature and washed with water and cold heptane to afford 0.27 g of 3-((tert-butyldimethylsilyl)oxy)abiraterone chlorhydrate (57% yield), free of impurities. Characteristic NMR signals:

¹H NMR (400 MHz, CDCl₃): δ 8.72 (s, 1H), 8.59 (s, 1H), 8.32 (d, J=7.4 Hz, 1H), 7.82 (s, 1H), 6.34 (s, 1H), 5.35 (d, J=5.1 Hz, 6H), 3.49 (s, 1H), 1.07 (s, 3H), 1.06 (s, 3H), 0.89 (s, 9H), 0.06 (s, 6H).

### d) Using More Catalyst and Base as Lowering the Temperature

In a round bottom flask under inert atmosphere were added consecutively: 0.57 g (1 mmol) of 3-((tert-butyldimethylsilyl)oxy)-5-dehydroepiandrosterone p-toluenesulfonyl hydrazone 13, 0.055 g (0.06 mmol, 6%) of Pd₂(dba)₃, 0.057 g (0.12 mmol, 12%) of XPhos and 0.6 g (7.5 mmol, 7.5 eq) of t-BuOLi. Then, 15 ml of dry dioxane were added to form a suspension with stirring, over which 0.14 ml (1.50 mmol, 1.5 eq) of 3-bromopyridine were added.

The reaction mixture was heated at 90° C. for 15 hours, and then it was cooled and filtered, washing the insoluble residue with THF (3×5 ml). The filtrates were combined and the solvent was evaporated under reduced pressure to afford a residue containing the desired product (65%) and a dimeric impurity resulting from two molecules of the starting material (6.4%).

The residue was solved in 10 ml of THF and 17 ml of HCl aq (1M) were added and a suspension was obtained. The solid obtained was filtered at room temperature, washing successively with water and cool hexane, to afford 0.3 g of 3-((tert-butyldimethylsilyl)oxy)abiraterone chlorhydrate (60% yield), with a purity by HPLC of around 95%.

### e) Using a Little Quantity of Water.

In a round bottom flask under inert atmosphere were added consecutively: 0.285 g (0.5 mmol) of 3-((tert-butyldimethylsilyl)oxy)-5-dehydroepiandrosterone p-toluenesulfonyl hydrazone 13, 0.0275 g (0.03 mmol, 6%) of Pd₂(dba)₃, 0.0280 g (0.06 mmol, 12%) of XPhos and 0.6 g (7.5 mmol, 15 eq) of t-BuOLi. Then, 15 ml of dry dioxane were added to form a suspension with stirring, over which 0.072 ml (0.75 mmol, 1.5 eq) of 3-bromopyridine were added. Finally 0.018 ml of water (1 equivalent) were also added.

The reaction mixture was heated at 90° C. for 7 hours, and then it was cooled and filtered, washing the insoluble residue with THF (3×5 ml). The filtrates were combined and the solvent was evaporated under reduced pressure to afford a residue containing the desired product (65%) and a dimeric impurity resulting from two molecules of the starting material (6.5%).

### Example 22

### Synthesis of 3-((tert-butyldimethylsilyl)oxy)abiraterone from 3-((tert-butyldimethylsilyl)oxy)-5-dehydroepiandrosterone 2,4,6-trimethylsulfonyl hydrazone

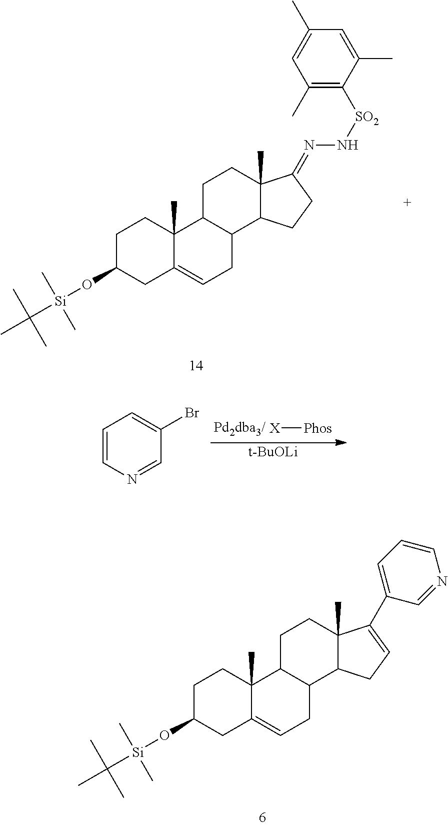

In a round bottom flask under inert atmosphere were added consecutively: 0.299 g (0.5 mmol) of 3-((tert-butyldimethylsilyl)oxy)-5-dehydroepiandrosterone 2,4,6-trimethylsulfonyl hydrazone 14, 0.0275 g (0.03 mmol, 6%) of Pd₂(dba)₃, 0.0275 g (0.06 mmol, 12%) of XPhos and 0.6 g (7.5 mmol, 15 eq) of t-BuOLi. Then, 7.5 ml of dry dioxane were added to form a suspension with stirring, over which 0.072 ml (0.75 mmol, 1.5 eq) of 3-bromopyridine and 0.018 ml of water were added.

The reaction mixture was heated at 90° C. for 6 hours, and then it was cooled and filtered, washing the insoluble residue with THF (3×5 ml). The filtrates were combined and the solvent was evaporated under reduced pressure to afford a residue containing the desired product (70%) and a dimeric impurity resulting from two molecules of the starting material of only 3.4%.

The residue was solved in 5 ml of THF and 10 ml of HCl aq (1M) were added and a suspension was obtained. The solid obtained was filtered at room temperature, washing successively with water and cool hexane, to afford 3-((tert-butyldimethylsilyl)oxy)abiraterone chlorhydrate with a purity by NMR of more than 95%.

### Example 23

### Synthesis of 3-((tert-butyldimethylsilyl)oxy)abiraterone from 3-((tert-butyldimethylsilyl)oxy)-5-dehydroepiandrosterone 2,4,6-tri-isopropylsulfonyl hydrazone

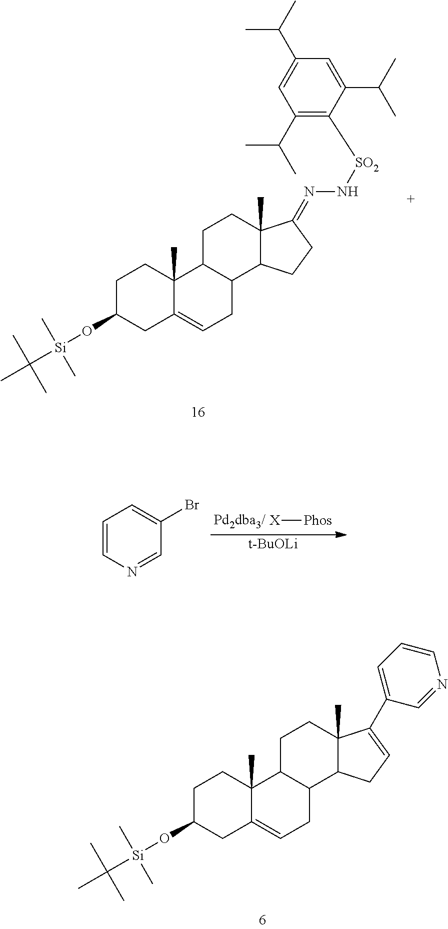

In a round bottom flask under inert atmosphere were added consecutively: 0.170 g (0.25 mmol) of 3-((tert-butyldimethylsilyl)oxy)-5-Dehydroepiandrosterone 2,4,6-tri-isopropylsulfonyl hydrazone 16, 0.014 g (0.015 mmol, 6%) of Pd₂(dba)₃, 0.014 g (0.03 mmol, 12%) of XPhos and 0.150 g (1.875 mmol, 7.5 eq) of t-BuOLi. Then, 3.75 ml of dry dioxane were added to form a suspension with stirring, over which 0.036 ml (0.375 mmol, 1.5 eq) of 3-bromopyridine were added.

The reaction mixture was heated at 110° C. overnight, and then it was cooled and filtered, washing the insoluble residue with THF (3×5 ml). The filtrates were combined and the solvent was evaporated under reduced pressure to afford a residue containing the desired product (60%) and a dimeric impurity resulting from two molecules of the starting material of 15.6%.

### Example 24

### Synthesis of abiraterone acetate from 3-acetyl-5-dehydroepiandrosterone toluenesulfonyl hydrazone

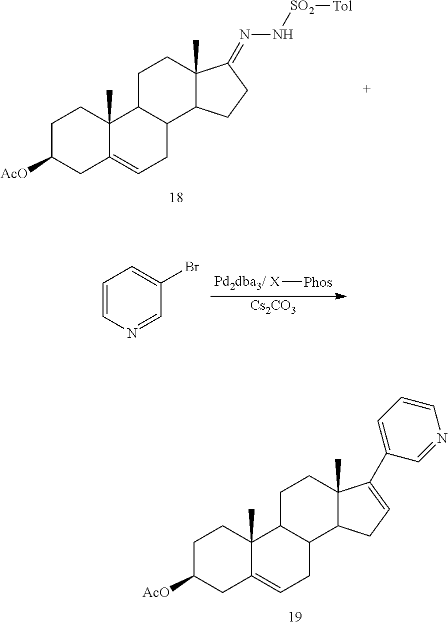

In a round bottom flask under inert atmosphere were added consecutively: 0.25 g (0.5 mmol) of 3-acetyl-5-dehydroepiandrosterone toluenesulfonyl hydrazone 18, 0.01 g (0.01 mmol, 2%) of Pd₂(dba)₃, 0.012 g (0.024 mmol, 5%) of XPhos and 0.36 g (2.2 eq) of Cs₂CO₃. Thereafter 7.5 ml of dry dioxane were added to afford a suspension with stirring and 0.072 ml (1.5 eq) of 3-bromopyridine were also added to the suspension. The reaction mixture was heated at 110° C. for 5 hours, and then it was cooled and filtered, washing the insoluble residue with THF (3×5 ml). The filtrates were combined and the solvent was evaporated under reduced pressure to afford a residue containing abiraterone acetate 19 (10%) and a dimeric impurity resulting from two molecules of the starting material (80%).

### Example 25

### Synthesis of abiraterone acetate from 3-acetyl-5-dehydroepiandrosterone toluenesulfonyl hydrazone

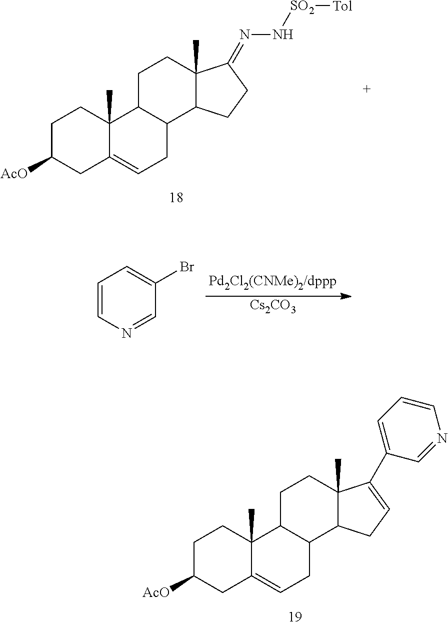

In a round bottom flask under inert atmosphere were added consecutively: 0.124 g (0.25 mmol) of 3-acetyl-5-dehydroepiandrosterone p-toluenesulfonyl hydrazone 18, 0.003 g (5%) of PdCl₂(CNMe)₂, 0.01 g (10%) of dppp and 0.24 g (3 eq.) of Cs₂CO₃. Thereafter 5 ml of dry dioxane were added to afford a suspension with stirring and 0.029 ml (1.5 eq) of 3-bromopyridine were also added to the suspension. The reaction mixture was heated at 90° C. for 52 hours, and then it was cooled and filtered, washing the insoluble residue with THF (3×5 ml). The filtrates were combined and the solvent was evaporated under reduced pressure to afford a residue containing abiraterone acetate 19 (15%) and a dimeric impurity resulting from two molecules of the starting material (42%).

### Example 26

### Synthesis of 3-((tert-butyldimethylsilyl)oxy)abiraterone from 3-((tert-butyldimethylsilyl)oxy)-5-dehydroepiandrosterone p-toluenesulfonyl hydrazone

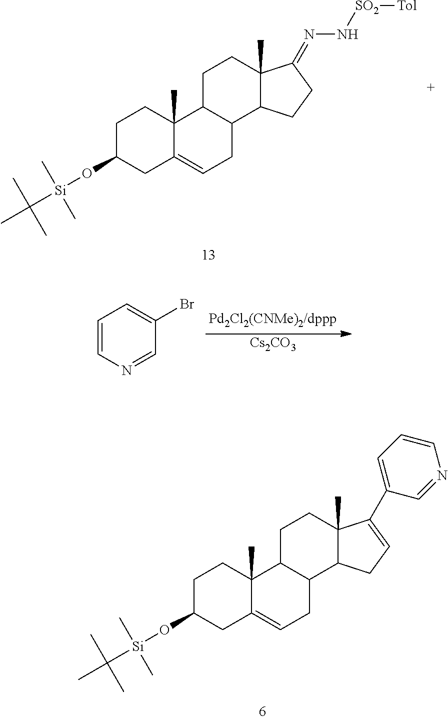

On a round bottom flask under inert atmosphere were added consecutively: 0.570 g (1 mmol) of 3-((tert-butyldimethylsilyl)oxy)-5-dehydroepiandrosterone p-toluenesulfonyl hydrazone 13, 0.013 g (5%) of PdCl₂(CNMe)₂, 0.041 g (10%) of dppp and 0.97 g (3 mmol) of Cs₂CO₃. Thereafter 15 ml of dry dioxane were added to afford a suspension with stirring and 0.144 ml (1.5 eq) of 3-bromopyridine were also added to the suspension.

The reaction mixture was heated at 110° C. overnight, and then it was cooled and filtered, washing the insoluble residue with THF (3×5 ml). The filtrates were combined and the solvent was evaporated under reduced pressure to afford a residue containing 3-((tert-butyldimethylsilyl)oxy)abiraterone 6 (31%) and a dimeric impurity resulting from two molecules of the starting material (38%).

### Example 27

### Synthesis of abiraterone from 3-((tert-butyldimethylsilyl)oxy)abiraterone

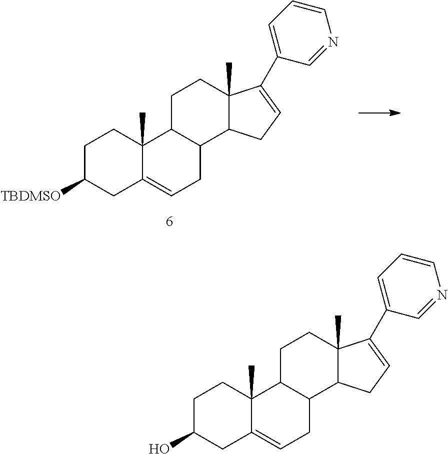

In a round bottom flask were dissolved 0.463 g (1 mmol) of 3-((tert-butyldimethylsilyl)oxy)abiraterone 6 in 10 ml of THF and 7.5 ml of a 1 Molar solution of tetrabutylammonium fluoride in THF was added. The reaction was monitored by TLC until complete deprotection of the hydroxyl group. Once the reaction was complete, the solvent was evaporated partially under reduced pressure and some water was added to the reaction mixture to afford a suspension. The solid formed was isolated by filtration, washed with water (3×5 ml) and dried under vacuum to afford 0.31 g (89% yield) of pure abiraterone, as confirmed by NMR.

### Example 28

### Synthesis of abiraterone chlorhydrate from 3-((tert-butyldimethylsilyl)oxy)-5-dehydroepiandrosterone P-toluenesulfonyl hydrazine

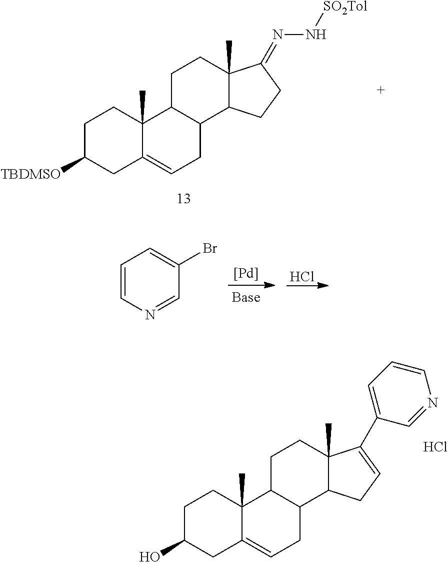

In a round bottom flask under inert atmosphere were added consecutively: 13 g (22.8 mmol) of 3-((tert-butyldimethylsilyl)oxy)-5-dehydroepiandrosterone p-toluenesulfonyl hydrazone 13, 0.78 g (1.35 mmol) of Pd₂(dba)₃, 1.3 g (2.72 mmol) of XPhos and 17.53 g (219 mmol, 9.5 eq) of t-BuOLi. Then, 195 ml of dioxane were added to form a suspension with stirring, over which 3.25 ml (34.2 mmol) of 3-bromopyridine and 0.39 ml (21.7 mmol) of water were added.

The reaction mixture was heated at reflux for 5 hours, and then it was cooled and filtered. Water (220 ml) and ethyl acetate (220 ml) were added over the filtrate and the two phases formed were decanted, the aqueous phase was extracted again with 100 ml of ethyl acetate and the organic phases were combined and evaporated under reduced pressure to afford a residue containing the desired product.

The residue was solved in 280 ml of isopropyl ether and 18.3 ml of HCl aq (10M) and 500 ml of water were added. A solid began to appear and a suspension was obtained. The solid obtained was filtered at room temperature, washing successively with water and cool heptane, to afford 6 g of abiraterone chlorhydrate (68% yield), with a purity by HPLC of around 95%.

### Example 29

### Synthesis of 3-((tert-butyldimethylsilyl)oxy)abiraterone clorhydrate

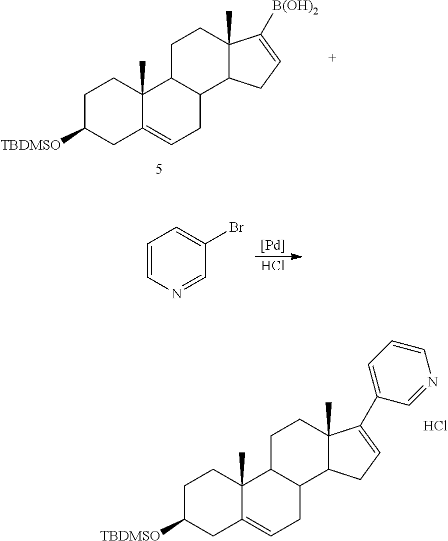

A solution of K₂CO₃ (0.92 g) in water (4.6 mL) under inert atmosphere was prepared. Compound 5 (2.5 g, 5.8 mmol) and dry THF (29 mL) were added. Solvents were deoxygenates and dichloro [1,1′-bis(diphenylphosphino)ferrocene] palladium(II)dichloromethane adduct (0.050 g) was added. Finally, 3-bromopyridine (0.7 ml) was added. The reaction mixture was refluxed for 15 h. The reaction mixture was cooled and filtered off. The liquid phase was washed with brine. Volatiles were evaporated under vacuum. The residue was solved in 25 ml of isopropyl ether and 1.8 ml of HCl aq (10M) and 125 ml of water were added. After 1 h stirring at room temperature a solid began to appear and a suspension was obtained. The solid obtained was filtered at room temperature and washed with water and cool heptane, to afford 2.61 g of 3-((tert-butyldimethylsilyl)oxy)abiraterone chlorhydrate (90% molar yield).

### Example 30

### Synthesis of 3-((tert-butyldimethylsilyl)oxy)abiraterone bromohydrate

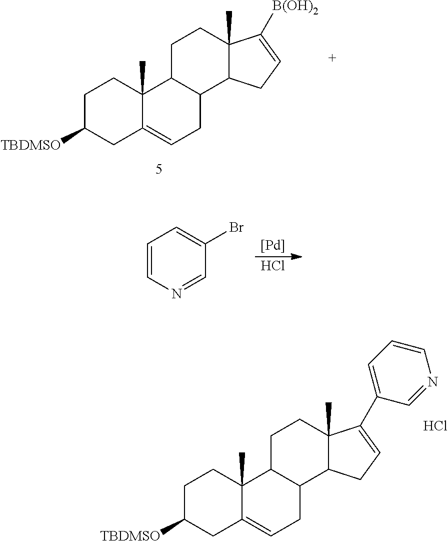

A solution of K₂CO₃ (0.92 g) in water (4.6 mL) under inert atmosphere was prepared. Compound 5 (2.5 g, 5.8 mmol) and dry THF (29 mL) were added. Solvents were deoxygenates and dichloro [1,1′-bis(diphenylphosphino)ferrocene] palladium(II)dichloromethane adduct (0.050 g) was added. Finally, 3-bromopyridine (0.7 ml) was added. The reaction mixture was refluxed for 17 h. The reaction mixture was cooled and filtered off. The liquid phase was washed with brine. Volatiles were evaporated under vacuum. The residue was solved in 25 ml of isopropyl ether and 3.7 ml of HBr 50% aq and 125 ml of water were added. After 1 h stirring at room temperature a solid began to appear and a suspension was obtained. The solid obtained was filtered at room temperature and washed with water and cool heptane, to afford 2.84 g of 3-((tert-butyldimethylsilyl)oxy)abiraterone bromhydrate (90% molar yield).

### Example 31

### Synthesis of 3-((tert-butyldimethylsilyl)oxy)abiraterone malate

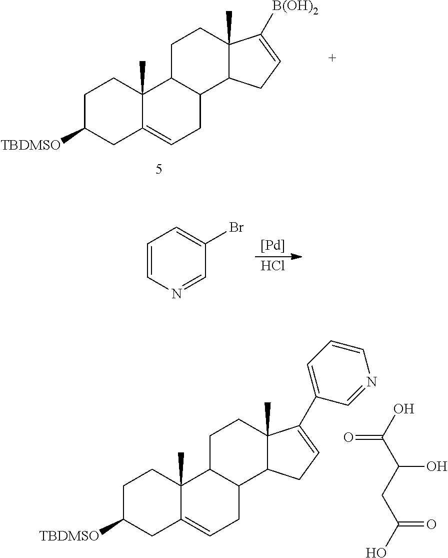

A solution of K₂CO₃ (0.92 g) in water (4.6 mL) under inert atmosphere was prepared. Compound 5 (2.5 g, 5.8 mmol) and dry THF (29 mL) were added. Solvents were deoxygenates and dichloro [1,1′-bis(diphenylphosphino)ferrocene] palladium(II)dichloromethane adduct (0.050 g) was added. Finally, 3-bromopyridine (0.7 ml) was added. The reaction mixture was refluxed for 18 h. The reaction mixture was cooled and filtered off. The liquid phase was washed with brine. Volatiles were evaporated under vacuum. The residue was solved in 25 ml of isopropyl ether and 0.09 g was added. After 1 h stirring at room temperature a solid began to appear and a suspension was obtained. The solid obtained was filtered at room temperature and washed with water and cool heptane, to afford 2.77 g of 3-((tert-butyldimethylsilyl)oxy)abiraterone malate (80% molar yield).

### Example 32

### Synthesis of abiraterone clorhydrate

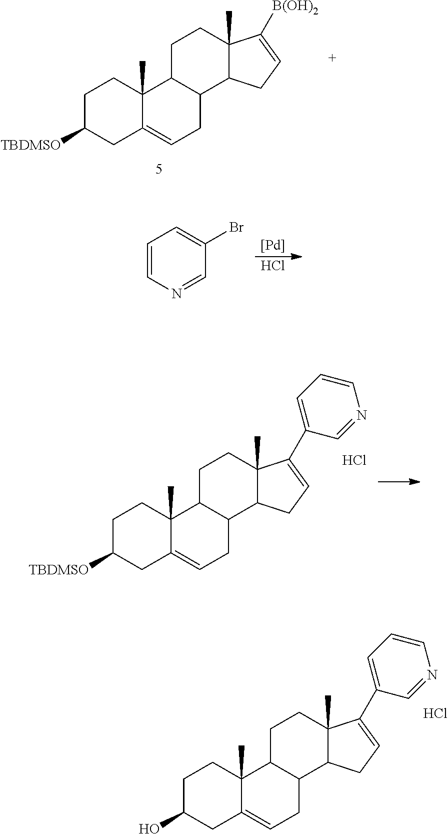

A solution of CaCO₃ (3.7 g) in water (18 mL) under inert atmosphere was prepared. Compound 5 (10.0 g, 23.2 mmol) and dry THF (50 mL) were added. Solvents were deoxygenates and dichloro [1,1′-bis(diphenylphosphino)ferrocene] palladium(II)dichloromethane adduct (0.15 g) was added. Finally, 3-bromopyridine (2.8 ml) was added. The reaction mixture was refluxed for 18 h. The reaction mixture was cooled water (42 mL) was added and the mixture was and filtered off. The liquid phase was washed with brine. Volatiles were evaporated under vacuum. The residue was solved in 60 ml of Ethyl acetate and 3.3 ml of HCl aq (10M) were added. After 1 h stirring at room temperature a solid began to appear and a suspension was obtained. The solid obtained was filtered off at room temperature and washed with water (50 mL). The wet cake was suspended with Methanol (60 mL) and 4.0 ml of HCl aq (10M) and stirred for 0.5 h at room temperature. Volatiles were concentrated under vacuum. The solid obtained was filtered off at room temperature and washed with water (30 mL) and dried under vacuum (50° C.) to afford 6.17 g of abiraterone chlorhydrate (69% molar yield).

### Example 33

### Synthesis of abiraterone clorhydrate

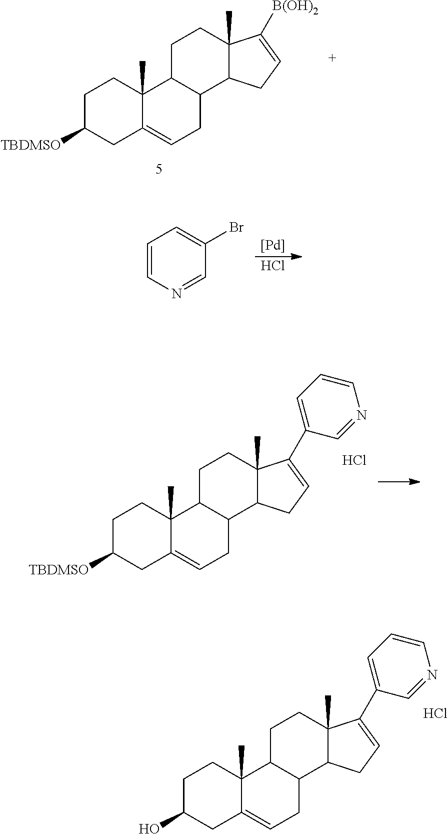

A solution of K₂CO₃ (0.37 g) in water (1.8 mL) under inert atmosphere was prepared. Compound 5 (1.0 g, 2.32 mmol) and water (5.0 mL) were added. Solvents were deoxygenates and dichloro [1,1′-bis(diphenylphosphino)ferrocene] palladium(II)dichloromethane adduct (0.015 g) was added. Finally, 3-bromopyridine (0.27 ml) was added. The reaction mixture was refluxed for 16 h. The reaction mixture was cooled water (4.2 mL) was added and the mixture was and filtered off. The liquid phase was washed with brine. Volatiles were evaporated under vacuum. The residue was solved in 6.0 ml of Ethyl acetate and 0.3 ml of HCl aq (10M) were added. After 1 h stirring at room temperature a solid began to appear and a suspension was obtained. The solid obtained was filtered off at room temperature and washed with water (50 mL). The wet cake was suspended with Methanol (6.0 mL) and 0.5 ml of HCl aq (10M) and stirred for 0.5 h at room temperature. Volatiles were concentrated under vacuum. The solid obtained was filtered off at room temperature and washed with water (3.0 mL) and dried under vacuum (50° C.) to afford 0.63 g of abiraterone chlorhydrate (70% molar yield).

What is claimed is:

1. A process for obtaining a compound of formula (I) 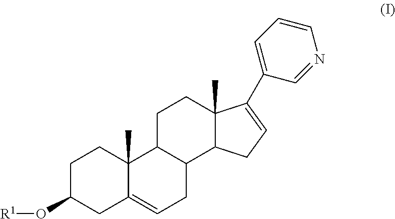
or a salt or solvate thereof, wherein
R¹ is selected from the group consisting of H and a hydroxyl protecting group; comprising reacting a compound of formula (IV) 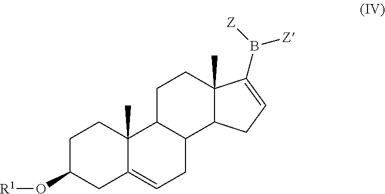
wherein
R¹ is selected from the group consisting of H and a hydroxyl protecting group; and
Z and Z′ are independently selected from the group consisting of hydroxyl, optionally substituted C₁-C₈ alkoxy and optionally substituted C₁-C₈ alkyl, or Z and Z′ together form an optionally substituted C₂-C₃ alkylenedioxy group or an optionally substituted C₆ aryldioxy group;
with a compound of formula (III) 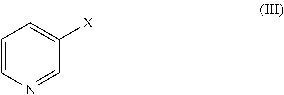
wherein
X is halogen or OSO₂CF₃.
in the presence of a palladium catalyst and a base.

2. The process according to claim 1, wherein the palladium catalyst is selected from Pd(PPh₃)₄, Pd₂(dba)₃, Pd(OAc)₂, Pd(PPh₃)₂Cl₂, Pd(dppe)₂Cl₂, Pd(dppf)Cl₂, Pd(dppf)Cl₂.CH₂Cl₂, Pd(dcypp)Cl₂, Pd(PhCN)₂Cl₂ and Pd(CH₃CN)₂Cl₂.

3. The process according to any claim 1, wherein the base is selected from alkaline and alkaline earth metal carbonates, bicarbonates, phosphates, acetates, alkoxides, hydroxides and halides.

4. The process according to claim 1, wherein the process is performed in the presence of a solvent or mixture of solvents selected from THF, toluene and water; or THF and water; or water.

5. The process according to claim 1, wherein the compound of formula (IV) is prepared by:
a) reacting a compound of formula (IIa) 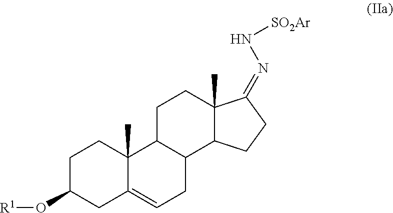
wherein
R¹ is selected from the group consisting of H and a hydroxyl protecting group; and
Ar is optionally substituted C₆-C₁₄ aryl;
with a lithium base and a compound of formula (VIII) 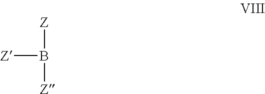
wherein
Z and Z′ are independently selected from the group consisting of hydroxyl, optionally substituted C₁-C₈ alkoxy and optionally substituted C₁-C₈ alkyl, or Z and Z′ together form an optionally substituted C₂-C₃ alkylenedioxy group or an optionally substituted C₆ aryldioxy group;
Z″ is selected from the group consisting of hydroxyl, optionally substituted C₁-C₈ alkoxy and optionally substituted C₁-C₈ alkyl;
or wherein the compound of formula (IV) is prepared by:
b) reacting a compound of general formula (IX) 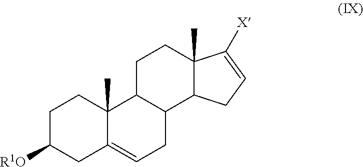
wherein
R¹ is selected from the group consisting of H and a hydroxyl protecting group; and
X′ is bromo or iodo;
with a lithium base and a compound of formula (VIII) 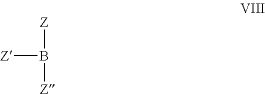
wherein
Z and Z′ are independently selected from the group consisting of hydroxyl, optionally substituted C₁-C₈ alkoxy and optionally substituted C₁-C₈ alkyl, or Z and Z′ together form an optionally substituted C₂-C₃ alkylenedioxy group or an optionally substituted C₆ aryldioxy group;
Z″ is selected from the group consisting of hydroxyl, optionally substituted C₁-C₈ alkoxy and optionally substituted C₁-C₈ alkyl.

6. A process for obtaining a compound of general formula (I) 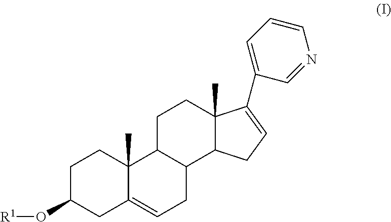
or a salt or solvate thereof,
wherein
R¹ is selected from the group consisting of H, and a hydroxyl protecting group; comprising reacting a compound of general formula (II) 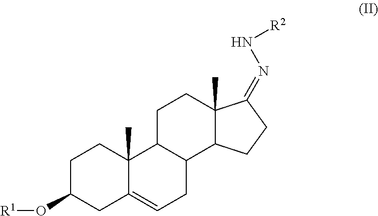
wherein
R¹ has the previously mentioned meanings, R² is SO₂R⁷ and R⁷ is selected from the group consisting of optionally substituted C₁-C₈ alkyl and optionally substituted C₆-C₁₄ aryl;
with a compound of formula (III) 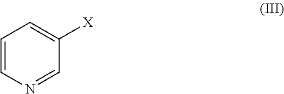
wherein
X is halogen or OSO₂CF₃;
in the presence of a palladium catalyst and a base.

7. The process according to claim 6, wherein the palladium catalyst is selected from Pd₂(dba)₃, Pd(PPh₃)₄, Pd(dppf)Cl₂.CH₂Cl₂, Pd(dcypp)Cl₂, PdCl₂(CNMe)₂, Pd(OH)₂ and Pd(OAc)₂.

8. The process according to claim 7, further comprising the addition of a ligand to the reaction media, said ligand being preferably selected from X-phos (2-dicyclohexylphosphino-2′,4′,6′-triisopropylbiphenyl), dppp (1,4-bis(diphenylphosphino)butane), S-phos (2-dicyclohexylphosphino-2′,6′-dimethoxybiphenyl), dppm (1,1-bis(diphenylphosphino)-methane), dippe (1,2-bis(diisopropylphosphino)ethane, dmpe (1,2-Bis(dimethylphosphino)ethane and dppe (1,2-bis(diphenylphosphino)ethane.

9. The process according to claim 6, wherein the base is selected from alkoxides and carbonates of alkaline and alkaline earth metals, preferably from t-BuOLi, MeOLi, MeONa and CsCO₃.

10. The process according to claim 6, wherein R² is selected from the group consisting of SO₂Ph, SO₂Tol, SO₂(2,4,6-trimethylphenyl) and SO₂(2,4,6-triisopropylphenyl).

11. The process according to claim 6, wherein the compound of general formula (II) is prepared by reacting a ketone of general formula (VI) 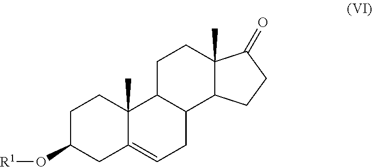
wherein
R¹ is selected from the group consisting of H and a hydroxyl protecting group; and a hydrazine of general formula (VII): 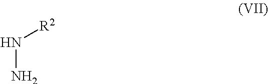
wherein
R² is SO₂R⁷ and R⁷ is selected from the group consisting of optionally substituted C₁-C₈ alkyl and optionally substituted C₆-C₁₄ aryl.

12. The process according to claim 6, wherein R¹ is selected from the group consisting of H, COMe, SitBuMe₂ (TBDMS) and SiPhMe₂ (DMPS).

13. The process according to claim 11, wherein R¹ is a silyl protecting group of formula Si(R³)(R⁴)(R⁵), wherein R³, R⁴ and R⁵ are independently selected from the group consisting of optionally substituted C₁-C₈ alkyl, optionally substituted C₃-C₆ cycloalkyl, optionally substituted C₆-C₁₄ aryl, optionally substituted C₁-C₈ alkoxy, and halogen.

14. The process according to claim 1, further comprising the purification of the compound of general formula (I) by means of crystallization and/or salt formation.

15. The process according to claim 1, further comprising the transformation of the compound of general formula (I) obtained in other compound of general formula (I), said transformation comprising one or more of the following steps:
i) transformation of the compound of formula (I) wherein R¹ is a hydroxyl protecting group into abiraterone (R¹═H), by means of a deprotection reaction which, depending on the nature of group R₁, comprises:
a) hydrolysis in acid or basic media,
b) use of fluoride reagents, or
c) oxidation or reduction;
ii) esterification of abiraterone (R¹═H) to afford abiraterone acetate (R¹═Ac).

16. An intermediate compound selected from:
a) a compound of formula (II) 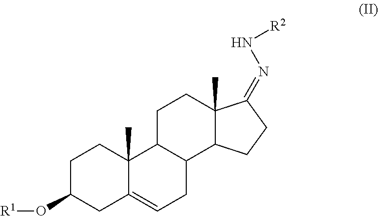
or a salt or solvate thereof, wherein
R¹ is selected from the group consisting of H and a hydroxyl protecting group;
R² is SO₂R⁷;
R⁷ is selected from the group consisting of optionally substituted C₁-C₈ alkyl and optionally substituted C₆-C₁₄ aryl;
with the proviso that the following compounds are not included: 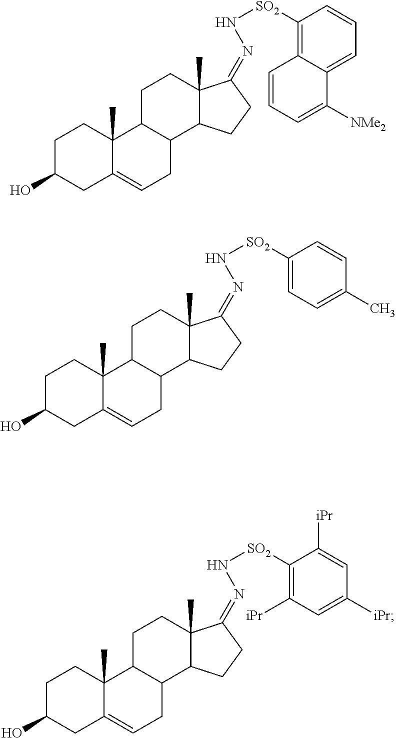
b) a compound of formula (IX) 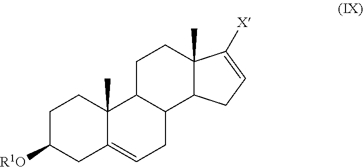
or a salt or solvate thereof, wherein
R¹ is a hydroxyl protecting group, and
X′ is bromo or iodo;
c) a compound of formula (V) 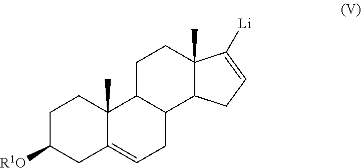
or a salt or solvate thereof, wherein
R¹ is a hydroxyl protecting group; and
d) a compound of formula (IV) 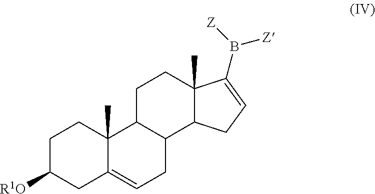
or a salt or solvate thereof, wherein
R¹ is selected from the group consisting of H and a hydroxyl protecting group; and
Z and Z′ are independently selected from the group consisting of hydroxyl, optionally substituted C₁-C₈ alkoxy and optionally substituted C₁-C₈ alkyl, or Z and Z′ together form an optionally substituted C₂-C₃ alkylenedioxy group or an optionally substituted C₆ aryldioxy.

17. A compound of formula (I) 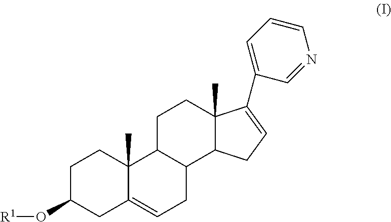
or a salt or solvate thereof,
wherein
R¹ is SiR³R⁴R⁵. and
R³, R⁴ and R⁵ are independently selected from the group consisting of optionally substituted C₁-C₈ alkyl, optionally substituted C₃-C₆ cycloalkyl, optionally substituted C₆-C₁₄ aryl, optionally substituted C₁-C₈ alkoxy, and halogen.

18. A compound of formula:

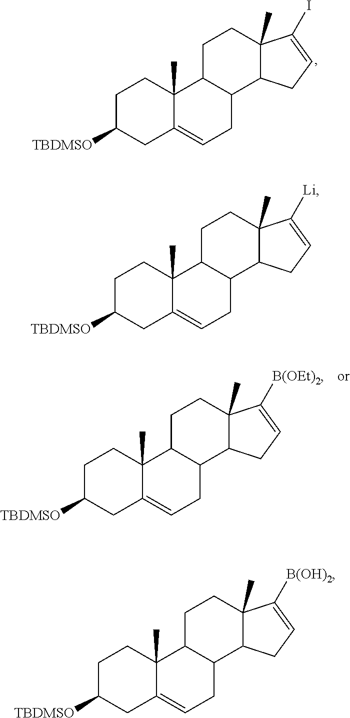

19. A process for the preparation of a salt of a compound of formula (I) as defined in claim 17 by recovering the salt from a solution of the free base in any suitable solvent by treating the solution with an appropriate acid, wherein preferably the compound of formula (I) is 3-TBDMS-abiraterone and/or the acid is hydrochloric acid so that the salt is the chlorhydrate salt.

20. The process according to claim 6, further comprising the purification of the compound of general formula (I) by means of crystallization and/or salt formation.

21. The process according to claim 6, further comprising the transformation of the compound of general formula (I) obtained in other compound of general formula (I), said transformation comprising one or more of the following steps:
iii) transformation of the compound of formula (I) wherein R¹ is a hydroxyl protecting group into abiraterone (R¹═H), by means of a deprotection reaction which, depending on the nature of group R₁, comprises:
a) hydrolysis in acid or basic media,
b) use of fluoride reagents, or
c) oxidation or reduction;
iv) esterification of abiraterone (R¹═H) to afford abiraterone acetate (R¹═Ac).
